(19) United States
(12) Patent Application Publication
(10) Pub. No.: US 2026/0226485 A1
(43) Pub. Date: Aug. 6, 2026

(54) METHODS FOR PRODUCING HIGH-LEVEL SQUALENE

(71) Applicant: Massachusetts Institute of Technology, Cambridge, MA (US)

(72) Inventors: Gregory Stephanopoulos, Winchester, MA (US); Yongshuo Ma, Quincy, MA (US)

(73) Assignee: Massachusetts Institute of Technology, Cambridge, MA (US)

(21) Appl. No.: 19/150,973

(22) PCT Filed: Jan. 25, 2024
(86) PCT No.: PCT/US2024/012930

§ 371 Date: Jul. 25, 2025

Related U.S. Application Data
(60) Provisional application No. 63/481,957, filed on Jan. 27, 2023.

(51) Int. Cl. C12N 15/81; C07K 14/255; C12N 1/16; C12N 9/00; C12N 9/02; C12N 9/10; C12N 9/20; C12N 15/52; C12P 5/00; C12R 1/645

(52) U.S. Cl. CPC C12N 15/81; C07K 14/255; C12N 1/16; C12N 9/0008; C12N 9/1029; C12N 9/20; C12N 9/93; C12N 15/52; C12P 5/007; C12R 2001/645; C12Y 102/01012; C12Y 203/0102; C12Y 203/03008; C12Y 301/01003; C12Y 602/01001; C12Y 604/01001; C12Y 604/01002

(57) ABSTRACT

Aspects of the present disclosure relate to modified yeast cells capable of producing increased quantities of squalene as compared to unmodified yeast cells and methods of using the same.

### CROSS-REFERENCE TO RELATED APPLICATIONS

This application claims the benefit under 35 U.S.C. § 119(e) of U.S. Provisional Application No. 63/481,957, filed Jan. 27, 2023, titled “METHODS FOR PRODUCING HIGH-LEVEL SQUALENE IN YARROWIA LIPOLYTICA,” the entire disclosure of which is hereby incorporated by reference in its entirety.

### GOVERNMENT LICENSE RIGHTS

This invention was made with government support under DE-SC0022016 awarded by U.S. Department of Energy. The government has certain rights in the invention.

### REFERENCE TO AN ELECTRONIC SEQUENCE LISTING

The contents of the electronic sequence listing (M065670542W000-SEQ-KVC.xml; Size: 71,323 bytes; and Date of Creation: Jan. 11, 2024) is herein incorporated by reference in its entirety.

### FIELD

The present disclosure relates to the modified yeast cells capable of producing increased quantities of squalene as compared to unmodified yeast cells and methods of use thereof.

### BACKGROUND

Squalene is a colorless organic triterpene with the molecular formula C₃₀H₅₀. It is a biochemical precursor to both steroids and hopanoids. Squalene may also be used as a vaccine adjuvant, and it is found in many commercial hair and topical skin products. Chemical synthesis of squalene is laborious and often results in low yields.

### SUMMARY

The present disclosure relates, at least in part, to modified yeast cells capable of producing increased quantities of squalene as compared to unmodified yeast cells and methods of using the same. Squalene is a commercially valuable organic compound originally derived from shark liver oil (Squalus is a genus of sharks). Naturally occurring squalene is difficult to obtain, and chemical synthesis of squalene is laborious and often results in low yields.

Accordingly, aspects of the present disclosure relate to a modified yeast cell, comprising: a first set of heterologous polynucleotides encoding a first set of enzymes of the mevalonate pathway, wherein one or more of the first set of enzymes of the mevalonate pathway is/are linked to a peroxisomal targeting signal; a first heterologous polynucleotide encoding a lipase; and a second set of heterologous polynucleotides encoding a second set of enzymes having triacylglycerol synthesis activity.

In some embodiments, the modified yeast cell further comprises a third set of heterologous polynucleotides encoding a third set of enzymes having acetyl-CoA synthesis activity.

In some embodiments, the modified yeast cell further comprises a fourth set of heterologous polynucleotides encoding a fourth set of enzymes having β-oxidation activity.

In some embodiments, the first set of enzymes of the mevalonate pathway comprises ERG10, ERG13, tHMGR, mvaE, mvaS, ERG12, ERG8, ERG19, IDI, ERG20, ERG9, or any combination thereof.

In some embodiments, each enzyme of the first set of enzymes of the mevalonate pathway comprises a peroxisomal targeting signal. In some embodiments, ERG20 and ERG9 each comprise a peroxisomal targeting signal. In some embodiments, the peroxisomal targeting signal is at the C-terminus of the one or more of the first set of enzymes of the mevalonate pathway. In some embodiments, the peroxisomal targeting signal is at the N-terminus of the one or more of the first set of enzymes of the mevalonate pathway. In some embodiments, the peroxisomal targeting signal is peroxisomal targeting sequence 1 (PTS1), peroxisomal targeting sequence 2 (PTS2), or peroxisomal targeting sequence 3 (PTS3). In some embodiments, the PTS1 is the amino acid sequence serine-lysine-leucine (SKL).

In some embodiments, the lipase is a triacylglycerol lipase derived from Thermomyces lanuginosus.

In some embodiments, the second set of enzymes having triacylglycerol synthesis activity comprises acetyl-CoA carboxylase (ACC1), diacylglycerol acyltransferase (DGA1), NAD+-dependent G3P dehydrogenase (GPD1), or any combination thereof.

In some embodiments, the third set of enzymes having acetyl-CoA synthesis activity comprises pyruvate carboxylase (PYC1), ATP:citrate lyase (ACL), citrate carrier YHM2, acetyl-CoA synthetase, or any combination thereof. In some embodiments, the ACL is derived from Mus musculus. In some embodiments, the acetyl-CoA synthetase is derived from Salmonella enterica. In some embodiments, the acetyl-CoA synthetase comprises an L641P amino acid substitution mutation relative to a wild-type acetyl-CoA synthetase.

In some embodiments, the fourth set of enzymes having β-oxidation activity comprises POX1, POX2, POX3, POX4, POX5, POX6, multifunctional β-oxidation protein (MFE1), 3-ketoacyl-CoA thiolase (POT1), or any combination thereof.

In some embodiments, the modified yeast cell is an oleaginous yeast cell. In some embodiments, the oleaginous yeast cell is a Yarrowia cell. In some embodiments, the Yarrowia cell is Yarrowia lipolytica.

Aspects of the present disclosure relate to a method of producing squalene, comprising: culturing any modified yeast cell described herein in a culture medium for a sufficient time to produce squalene in the peroxisome of the modified yeast cell.

In some embodiments, the method cell further comprises extracting squalene from the modified yeast cell culture. In some embodiments, the culture medium comprises yeast extract, peptone, glucose, and an aqueous buffer. In some embodiments, the culture medium comprises acetate. In some embodiments, the culture medium comprises acetate and a limited amount of one or more sugars. In some embodiments, the one or more sugars comprise glucose, fructose, and/or xylose. In some embodiments, the culture medium comprises a buffer, optionally wherein the buffer is phosphate-buffered saline.

In some embodiments, the sufficient time is at least 6 hours, at least 12 hours, at least 24 hours, at least 48 hours, at least 72 hours, at least 96 hours, or at least 192 hours.

In some embodiments, at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is in the culture medium. In some embodiments, at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is extracted from the modified yeast cell culture. In some embodiments, at least at least 25 mg/g dry cell weight (DCW), at least 50 mg/g DCW, at least 100 mg/g DCW, at least 150 mg/g DCW, or more than 150 mg/g DCW of squalene is extracted from the modified yeast cell culture.

Further, this Summary introduces a selection of concepts in simplified form that are described further below in the Detailed Description. This Summary neither identifies key or essential features, nor limits the scope, of the claimed subject matter.

One aspect of the disclosure herein is a method of producing squalene in a yeast, the method comprising
a. providing an oleaginous yeast;
b. introducing modifications in the oleaginous yeast to elevate squalene production, wherein the modifications comprise
   i. introducing genes encoding the mevalonate pathaway and its downstream squalene synthesis pathway into the peroxisome of the yeast;
   ii. introducing a gene encoding the heterologous lipase from Thermomyces lanuginosus (tlTGF); and
   iii. overexpressing genes encoding enzymes responsible for triacylglycerol synthesis;
c. growing the modified yeast in a medium with a carbon source;
d. harvesting the squalene from the peroxisomes of the modified yeast.

In one embodiment of the disclosed method, the modifications introduced into the oleaginous yeast further comprise
i. upregulating the ATP:citrate lyase (ACL)-based cytosolic acetyl-CoA formation pathway, and
ii. overexpressing pyruvate carboxylase PYC1, MmACL from Mus musculus, and mitochondrial carrier YHM2.

In one embodiment of the disclosed method, the oleaginous yeast is Yarrowia lipolytica.

In one embodiment of the disclosed method, the medium comprises a yeast extract, peptone, glucose and an aqueous buffer.

In one embodiment of the disclosed method, the medium comprises acetate.

T In one embodiment of the disclosed method, squalene production was more than 0.5 g/L, more than 1.0 g/L, or more than 1.5 g/L.

In one embodiment of the disclosed method, squalene production was more than 50 mg/g DCW, more than 100 mg/g DCW, or more than 150 mg/g DCW.

In one embodiment of the disclosed method, squalene production was more than 0.5 g/L, more than 1.0 g/L, or more than 1.5 g/L.

In one embodiment of the disclosed method, squalene production was more than 50 mg/g DCW, more than 100 mg/g DCW, or more than 150 mg/g DCW.

In one embodiment of the disclosed method, the growing the modified yeast in a medium with a carbon source comprises cultivating the cells for at least 24 h, at least 48 hours; at least 72 hours, at least 96 hours, or at least 192 hours.

One aspect of the disclosure herein is an oleaginous yeast modified by
a. introducing genes encoding the mevalonate pathaway and its downstream squalene synthesis pathway into the peroxisome of the yeast;
b. introducing a gene encoding the heterologous lipase from Thermomyces lanuginosus (tlTGF); and
c. overexpressing genes encoding enzymes responsible for triacylglycerol synthesis.

In one embodiment, the disclosed oleaginous yeast is further modified by
a. upregulating the ATP:citrate lyase (ACL)-based cytosolic acetyl-CoA formation pathway, and
b. overexpressing pyruvate carboxylase PYC1, MmACL from Mus musculus, and mitochondrial carrier YHM2.

In one embodiment of the disclosed oleaginous yeast, yeast is Yarrowia lipolytica.

The following Detailed Description references the accompanying drawings which form a part this application, and which show, by way of illustration, specific example implementations. Other implementations may be made without departing from the scope of the disclosure.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF THE DRAWINGS

The following drawings form part of the present specification and are included to further demonstrate certain aspects of the present disclosure, which may be better understood by reference to one or more of the drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIGS. 1A-1E show engineering the cytosolic metabolic pathway for squalene production. FIG. 1A shows a schematic view of metabolic pathway for squalene production in cytosol. Squalene biosynthetic pathway started from acetyl-CoA was divided into three modules: Up-pathway, Mid-pathway and Down pathway. ERG10, Acetyl-CoA acetyltransferase. ERG13, 3-hydroxy-3-methylglutary-CoA (HMG-CoA) synthase. tHMGR, truncated HMG-CoA reductase. ERG12, Mevalonate kinase. ERG8, Phosphomevalonate kinase. ERG19, Mevalonate pyrophosphate decarboxylase. IDI, IPP:DMAPP isomerase. ERG20, Farnesyl pyrophosphate synthetase. ERG1, Squalene synthase. MvaE and MvaS, from Enterococcus faecalis. IPP, Isopentenyl diphosphate. DMAPP, Dimethylallyl diphosphate. FPP, Farnesyl pyrophosphate. (FIG. 1B) shows each module was overexpressed in Y. lipolytica, individually and in combination. Squalene titers were measured after 48 hours of fermentation. (FIGS. 1C and 1D) shows squalene titers (FIG. 1C) and content (FIG. 1D) were significantly enhanced when YPD medium was supplemented with 0.2 M PBS. Samples in uncontained PBS medium were measured at 48 hours cultivation, whereas samples in contained PBS medium were measured at 72 hour cultivation. (FIG. 1E) shows fermentation time-course profiles indicated that PBS-added fermentation reached the highest squalene titers at 72 hour cultivation, yet, at 48 hours in the PBS-unconditioned fermentation. Statistical significance was tested using the two-sided Student's t-test, *P<0.05, **P<0.01, ***P<0.001. All data are represented as mean±SD of three biologically independent experiments.

FIGS. 2A-2C show construction of a peroxisomal squalene-synthesizing pathway in Y. lipolytica. FIG. 2A shows a schematic illustration of the peroxisomal orthogonal metabolic network for squalene production. Conventional production of squalene in Y. lipolytica relies on the endogenous cytosolic MVA pathway (shown in black), which tightly couples with sterol synthesis. Here, an orthogonal pathway was built for squalene synthesis in peroxisome by introducing the complete squalene pathway starting acetyl-CoA (shown in black), in which MVA pathway was assembled to harvest peroxisomal acetyl-CoA. FIG. 2B shows the entire squalene pathway was sequentially assembled in peroxisome or overexpressed in cytosol, respectively. Squalene titers were measured after 72 hours of fermentation. FIG. 2C shows a time-course profiles of squalene content and glucose concentration in medium in strain Sq06 with cytosolic overexpressed pathway and in strain Sq10 with peroxisomal engineered pathway. Statistical significance was tested using the two-sided Student's t-test, ***P<0.001. ns, not significant. All data are represented as mean±SD of three biologically independent experiments.

FIGS. 3A-3D show transforming cellular TAGs into squalene synthesis via lipid metabolism. FIG. 3A shows a schematic illustration of the intracellular lipid metabolism, including biosynthesis, hydrolysis and degradation. Overexpressed genes in this study are shown in black. PYC1, pyruvate carboxylase. YHM2, mitochondrial citrate carrier. MmACL, ATP:citrate lyase from Mus musculus. ACC1, acetyl-CoA carboxylase. GPD1, NAD⁺-dependent G3P dehydrogenase. DGA1, diacylglycerol acyltransferase. tlTGL, lipase from Thermomyces lanuginosus. POX1-6, acyl-CoA oxidases. MFE1, Multifunctional β-oxidation protein. POT1, 3-ketoacyl-CoA thiolase. PEX10, peroxisome biogenesis factor. FIG. 3B shows an enhancement of lipid biosynthesis and its hydrolysis through overexpressing corresponding rate-limiting enzymes potentially improved squalene production in peroxisome. FIG. 3C shows strengthening β-oxidation pathway into peroxisomal acetyl-CoA formation further enhanced squalene production. FIG. 3D shows fermentation profiles of squalene-producing strain Sq27 in a 3-L bioreactor. Statistical significance was tested using the two-sided Student's t-test, **P<0.01, ***P<0.001. ns, not significant. All data are represented as mean±SD of three biologically independent experiments.

FIGS. 4A-4I show establishing acetate utilization pathway in peroxisome for enhancement of acetyl-CoA supply. FIG. 4A shows a schematic illustration of the metabolic network based on acetate uptake. The arrows accompanied with a star indicate the orthogonal squalene synthetic pathway original from acetate in peroxisome. The gray arrows indicate supplemented small fraction of glucose in substrate cofeeding system mainly support cell growth. SeACS, acetyl-CoA synthetase from Salmonella enterica. FIG. 4B shows profiles of squalene production when stain Sq10 was grown in YPD or YPA medium with or without PBS. FIGS. 4C and 4D show a decreased biomass was observed when strain Sq10 cultured in YPA medium (FIG. 4C), compared to that in YPD medium, whereas squalene content in both YPA and YPD medium is same (FIG. 4D). FIGS. 4E and 4F show an introduction of SeACS (SeACS^{L641P}) in peroxisome constituted an orthogonal acetate utilization pathway in strain Sq28, which resulted in significant improvement of squalene production in terms of titers (FIG. 4E) and cellular content (FIG. 4F), compared to the parent strain Sq10. FIG. 4G shows acetic acid in both salt and acid forms as carbon source was implemented in acetate fermentation in bioreactor. As glucose batch feeding suppresses acetate consumption, glucose was continuously supplemented in small quantities to the acetate culture in cofeeding systems. FIGS. 4H and 4I shows fermentation profiles of squalene-producing strain Sq28 on acetate-only culture (FIG. 4H) and on glucose-acetate mix cofeeding culture (FIG. 4I). Statistical significance was tested using the two-sided Student's t-test, ***P<0.001. ns, not significant. All data are represented as mean±SD of three biologically independent experiments.

FIG. 5 shows the disruption of LYS5 in Y. lipolytica poIf strain via Crispr-Cas9. A single thymine insertion (triangle) caused a frameshift mutation (star) which abolished LYS5 activity.

FIG. 6 shows profiles of pH change in the YPD medium with or without PBS growing strain Sq06 over the fermentation course. All data are represented as mean±SD of three biologically independent experiments.

FIG. 7 shows the comparison of biomass between strain Sq03 overexpressing ERG20 and ERG9 in cytosol and strain Sq07 overexpressing ERG20 and ERG9 in peroxisome after 3 days of fermentation. All data are represented as mean±SD of three biologically independent experiments.

FIG. 8 shows squalene production of engineered strains with overexpression of endogenous lipases. Squalene titers were measured after 72 hours of fermentation. All data are represented as mean±SD of three biologically independent experiments.

FIG. 9 shows intracellular lipid production of engineered strains. Both engineered lipid biosynthetic pathway and optimized cytosolic acetyl-CoA flux significantly improved the intracellular lipid production. Statistical significance was tested using the two-sided Student's t-test, ***P<0.001. All data are represented as mean±SD of three biologically independent experiments.

FIGS. 10A-10B show cytosolic engineered squalene production after optimizing lipid pathway. When overexpression of three key enzymes ACC1, DGA1, GPD1 involved lipid biosynthesis in cytosolic engineered squalene-producing strain Sq06, a decreased squalene production was observed (FIG. 10A), despite a higher intracellular lipid level (FIG. 10B). Statistical significance was tested using the two-sided Student's t-test, ***P<0.001. All data are represented as mean±SD of three biologically independent experiments.

FIGS. 11A-11B show fine-tuning of NADPH generation for squalene production. Production of squalene (FIG. 11A) and intracellular lipid (FIG. 11B) when overexpressing of G6PD and 6PGD in engineered strains. Statistical significance was tested using the two-sided Student's t-test. ns, not significant. All data are represented as mean±SD of three biologically independent experiments.

FIG. 12 shows the comparison of citrate level when overexpressing PYC1 in the engineered strains. Citrate production was measured after 48 hours of cultivation. All data are represented as mean±SD of three biologically independent experiments.

FIGS. 13A-13B show squalene production in the engineered strains with enhanced β-oxidation pathway. Further overexpression of POX2, MFE1, POT1 and PEX10 in strain Sq28 with orthogonal acetate utilization pathway in peroxisome resulted in marginal improvement in squalene production, regardless of titer (FIG. 13A) and content (FIG. 13B). Statistical significance was tested using the two-sided Student's t-test, *P<0.05. ns, not significant. All data are represented as mean±SD of three biologically independent experiments.

### DETAILED DESCRIPTION

Engineering microorganisms for the production of high-value compounds from renewable feedstock is a promising alternative to replacement of traditional plant or fossil-dependent production (1-4). However, synthetic biology efforts to achieve economically viable titers and productivities are frequently hindered by native competition pathways and metabolic cross-talk (3). This is because the metabolic networks in host have evolved to be tightly regulated to maintain metabolic homeostasis, frequently making it particularly challenging to redirect metabolic fluxes toward pathways of interest (5). Although blocking competing pathways by inactivating competing genes (6) or utilizing inducible (7, 8) and weaken promoters (9) is a common strategy, it is still challenging when the biosynthetic pathway of interest competes with endogenous pathways that are essential for cell growth, prompting efforts to develop more effective approach to funnel metabolic fluxes into the target products. An alternative solution to overcome this obstacle would be to make the pathway of interest less connected to the native competing metabolism, which ideally must be orthogonal to the host metabolic network (10, 11). Reprogramming cellular metabolism and establishing an orthogonal route would be advantageous for improved pathway control and performance.

A characteristic case, where the establishment of an orthogonal metabolic pathway would be beneficial, is the synthesis of triterpenoids in yeast, in which their precursors is tightly coupled to the sterol biosynthetic pathway. Triterpenoids are one of the largest and most structurally diverse families of natural products, and many have been shown to possess potential use in the food industry, cosmetic, and pharmacology (12, 13). Among these, squalene is a linear triterpene oil widely used as dietary supplement, moisturizer and anti-tumor agent in pharmaceutical, health care, and cosmetic industries (14). In particular, squalene has been being employed as an essential component of nanoemulsion vaccine adjuvants in seasonal influenza and COVID-19 vaccines (15). Furthermore, squalene acts as a key precursor for synthesis of all structure-complexity triterpenoids and sterols in nature (16). Currently, its commercial production mainly comes from the shark liver oil and plant seeds (17). Due to a sizeable and rapidly expanding world market, sourcing this molecule from nature sources is no longer sustainable, and urgently require alternatives for squalene production with low-cost as well as sustainable. Previously, manipulations of central carbon metabolism and MVA pathway, downregulation of competing pathway and co-factor regeneration have achieved squalene overproduction with improved productivity in many microbial strains such as Saccharomyces cerevisiae (9, 18-21), Escherichia coli (22-24), Yarrowia lipolytica (25, 26) and other microorganisms (14). However, both production titer and yield necessitate further enhancement to enable industrial scale production that is economically feasible, with properly preventing squalene diversion from competing pathway being a major challenge.

Squalene is a linear triterpene oil widely distributed in plants, fungi, animals, and human body, and acts as a key precursor in the biosynthesis of diverse triterpenoids and steroids. Recently, it has found uses in the fields of pharmaceuticals, health products, and personal care. Squalene is an essential component of nanoemulsion vaccine adjuvants and can stimulate and enhance immunological response against antigens. It is most notably used in the well-known MF59 adjuvant, composed of 2.5% (v/v) squalene, 0.25% (w/v) Tween-80, and 0.25% (w/v) Span-85, widely administered in seasonal influenza, malaria, and Covid-19 vaccines. Squalene was initially characterized as obtained from shark liver oil in 1916, and for decades, the main natural source of squalene was from deep-sea shark livers. Due to overfishing and increased pollution in the ocean, shark-derived squalene is an unfavorable or unsustainable source for commercial application. Plant seed oil is an alternative source of squalene, which, however, is not an environmentally acceptable or economical method of producing squalene with high purity because of its low content and complex chemical mixtures inherent in plant extracts.

Synthetic squalene sourced from cultivation of microorganisms specifically engineered for its production has been evaluated for various application and found to be an equally efficacious and safe vaccine adjuvant to animal- and plant-sourced squalene. These positive results encouraged efforts to develop a microbe-producing platform via synthetic biology to replace traditional squalene production from shark liver oil and seeds. Although several microorganisms have been genetically engineered to overproduce squalene, both production titer and yield of these processes have been low and uneconomical for industrial scale production. One reason is that the squalene-synthesizing pathway competes for intracellular intermediates with other pathways vital for cell growth like sterol synthesis. Thus, in yeast, squalene synthesis in the cytosol is tightly coupled to the native sterol biosynthesis pathway, which is essential for cell growth. Because of this competition for sterol synthesis, it is challenging to engineer yeast where squalene synthesis is favored too much as this will lead to reduced sterol synthesis and growth impairment. This makes it very difficult to construct robust strains with high squalene productivity.

To overcome the limitation of squalene productivity in yeast, a complete pathway for squalene synthesis (the mevalonate pathway and its downstream squalene synthesis pathway) in the peroxisome of Yarrowia lipolytica was introduced. Peroxisomes are intracellular compartments that generate, via fatty acid degradation, the acetyl-CoA pool that can be harvested by a peroxisomal orthogonal squalene synthesis pathway and can also serve as storage compartments for squalene accumulation. A peroxisomal orthogonal pathway efficiently insulates locally formed squalene from competing cytosolic pathways, thus avoiding intricate regulatory mechanisms present in the cytosol. This strategy resulted in remarkable increase of squalene production.

It was also found that insufficient supply of the precursor acetyl-CoA in the peroxisome limited the productivity of squalene. Y. lipolytica is a genetically tractable oleaginous yeast capable of naturally accumulating large amounts of intracellular lipids. To overcome the acetyl-CoA limitation, the second part of the strategy comprised utilizing the accumulated lipid in Y. lipolytica for conversion into peroxisomal acetyl-CoA. To this end, the heterologous lipase tlTGL from Thermomyces lanuginosus targeted specifically to the lipid bodies was introduced. This lipase enzyme mediates hydrolysis of triacylglycerides (TAG) into free fatty acids, which are further degraded into acetyl-CoA via the β-oxidation pathway in the peroxisome. The expression of tlTGL thus accelerated lipid metabolism and led to significant increase of squalene production by virtue of an increased Acetyl-CoA supply.

To further improve substrate availability for squalene synthesis in the peroxisome, the key enzymes responsible for TAG synthesis were overexpressed, including acetyl-CoA carboxylase (ACC1), diacylglycerol acyltransferase (DGA1), and NAD⁺-dependent G3P dehydrogenase (GPD1). Their overexpression led to significant increase in the lipid content, which elevated considerably squalene production. Additionally, the cytosolic Acetyl-CoA synthesis pathway was upregulated by upregulating the ATP:citrate lyase (ACL)-based cytosolic acetyl-CoA formation pathway, and overexpressing pyruvate carboxylase PYC1, MmACL from Mus musculus, and mitochondrial carrier YHM2, which has been proposed to play an important role in lipid accumulation in oleaginous yeasts. These modulations further elevated the production of lipids and their conversion to squalene. Finally, the β-oxidation pathway was strengthened through overexpressing acyl-CoA oxidases (POX), Multifunctional β-oxidation protein (MFE1), and 3-ketoacyl-CoA thiolase (POT1). Up-regulation of the β-oxidation pathway accelerated fatty acid degradation releasing more acetyl-CoA for enhancement of squalene production in the peroxisome.

The combined result of these genetic modulations was that the peroxisomal orthogonal pathway was advantageously repurposed for build-up of squalene without impacting negatively cytosolic sterol biosynthesis, thus maintaining robust cell viability. Ultimately, a strain capable of producing 26.3 g/L squalene at a productivity of 0.144 g/L/h in bioreactor fermentations was established, the highest reported by microbial fermentation.

In summary, disclosed herein are metabolic modulations for squalene super-production by:
Introducing a complete pathway for squalene synthesis (including the mevalonate pathway and its downstream squalene synthesis pathway) in the peroxisome of Yarrowia lipolytica.
Introducing the heterologous lipase tlTGL from Thermomyces lanuginosus targeted specifically to the lipid bodies in order to mediate TAG hydrolysis into free fatty acids and increase of acetyl-CoA via the β-oxidation in the peroxisome.
Overexpressed the key enzymes responsible for TAG synthesis, including acetyl-CoA carboxylase (ACC1), diacylglycerol acyltransferase (DGA1), and NAD⁺-dependent G3P dehydrogenase (GPD1).
Enhancing the cytosolic acetyl-CoA synthesis pathway by upregulating the ATP:citrate lyase (ACL)-based cytosolic acetyl-CoA formation pathway, and overexpressing pyruvate carboxylase PYC1, MmACL from Mus musculus, and mitochondrial carrier YHM2. These modulations further elevated the production of lipids and their conversion to squalene.
Overexpressing acyl-CoA oxidases (POX), Multifunctional β-oxidation protein (MFE1), and 3-ketoacyl-CoA thiolase (POT1) in order to strengthen the beta oxidation pathway.

In this work, using the oleaginous yeast Y. lipolytica for squalene overproduction, the possibility of reprogramming an orthogonal metabolic network in peroxisome to circumvent metabolic competitions in triterpene synthesis was systematically explored. Two independent strategies that effectively enhance the precursor supply in peroxisome for triterpene production were also demonstrated. In yeast, squalene synthesis in cytosol is tightly coupled to the sterol biosynthetic pathway that is essential for cell growth and viability. This strong metabolic crosstalk leads to a major challenge in constructing squalene robust strains with high productivity. To address this issue, an orthogonal pathway in peroxisome was constructed by introducing a complete squalene synthetic pathway starting from acetyl-CoA, in which the mevalonate (MVA) pathway is assembled such as to harvest peroxisomal acetyl-CoA pool. Peroxisomal pathway efficiently insulates locally formed squalene from cytosolic competing pathway, as well as avoiding intricate regulatory mechanisms exists in cytosol, which in turn results in a remarkable increase of squalene production. However, the insufficient precursor acetyl-CoA supply in peroxisome is observed as bottleneck for peroxisomal production. In light of this, the first strategy was to transform the accumulated cellular lipid into peroxisomal acetyl-CoA pool through engineering lipid metabolism involved in biosynthesis, hydrolysis and degradation. This finally allows a peroxisomal producing strain capable of yielding 32.8 g/L squalene from glucose as carbon source with a 0.15 g/L/h productivity in bioreactor fermentations. Alternatively, in the second approach, similar production of squalene is obtained via establishing an orthogonal acetyl-CoA shortcut in peroxisomes by harnessing an acetate utilization pathway. This finally makes the peroxisomal production of squalene reach 31.6 g/L from acetate as primary carbon source via substrate co-feeding approach, with a 0.14 g/L/h productivity in bioreactor fermentations. Overall, the findings provide an efficient approach for the peroxisomal production of acetyl-CoA derived chemicals.

Engineering microorganisms for the production of valuable chemicals often requires competing intermediates and/or precursors with endogenous metabolic network to redirect high flux toward the product of interest. This is challenging, as millions of years of evolution have developed tightly regulated metabolic systems for optimal growth in the organism's natural environments (46). Therefore, it is imperative to develop methods that allow us to prevent metabolic precursors or intermediates from competing pathways and funnel carbon fluxes into the product of interest. Although blocking or downregulating competition pathways by deleting or weaken corresponding genes is a common strategy, it is still challenging to disrupt the host metabolic flux essential for cell viability.

Ideally, the heterologous pathway must be orthogonal to the host pathways (10), if the aim is to circumvent the competitions with chassis' native metabolism without affecting cell growth and viability. In this study, the Y. lipolytica was chosen as microbial host to explore the possibility of establishing an orthogonal metabolic network in peroxisome for the production of squalene, a representative of triterpenoids. A peroxisomal orthogonal pathway enabled a many-fold higher production of squalene than that of the cytosolic pathway, where squalene accumulation is suffering from strong sterol competition pathway. The higher productivity for squalene build-up in peroxisomes suggested that peroxisomes as barrier efficiently insulate the locally formed squalene from cytosolic competing pathway. This in turn transforms peroxisomes into microfactories for squalene build-up with less interruption to cell metabolism for growth. Although a peroxisome engineering strategy was widely applied in conventional model yeast S. cerevisiae for monoterpene production (28), its full potential in the oleaginous yeast Y. lipolytica remains largely unexplored for terpenoid biosynthesis, especially for triterpenes. On the other hand, due to very low intracellular lipid levels in S. cerevisiae, the generation of acetyl-CoA in peroxisomes for terpenoid synthesis is extremely limited. On the contrary, the oleaginous yeast Y. lipolytica is a natural lipid producer, potentially providing the relative higher acetyl-CoA pool for peroxisomal production. This advantage would make Y. lipolytica as an alternative host for peroxisome engineering production.

The acetyl-CoA supply in the peroxisome, however, was observed to be a bottleneck for further peroxisomal production. Considering Y. lipolytica's capacity of cellular lipid accumulation, insufficient acetyl-CoA supply could be overcome through promoting lipid degradation. While lipid bodies in Y. lipolytica are generally used as storage compartments for lipophilic compound sequestration, and often engineered for chemical elevation (36), enhanced lipid supply comes at the expense of acetyl-CoA, thereby limiting the flux into the MVA and product-forming pathway. Contrary to this traditional approach, in the present disclosure peroxisomes are advantageously repurposed as sites where squalene is synthesized, while at the same time acting as dynamic depots for locally formed squalene storage. This design avoids the concerns on how to balance the flux distribution between terpenoid and lipid synthesis. By reconverting the accumulated lipid, it was possible to supply a larger portion of the acetyl-CoA pool in peroxisomes for squalene production, and largely improve the carbon-utilizing efficiency, achieving higher titers and per-cell content. Interestingly, through lipid metabolism (biosynthesis, hydrolysis, and degradation), the transformation from cytosolic acetyl-CoA pool into peroxisomal acetyl-CoA pool was achieved.

In the second approach, construction of an acetyl-CoA shortcut in peroxisomes through an acetate utilization pathway greatly simplified supply of precursor and avoided the negative feedback regulation in host metabolism. In particular, the introduction of this precursor supply route was connected to the peroxisomal squalene pathway, which is completely uncoupled from native metabolism network, circumventing the competitions and metabolic crosstalk. Although acetate-only fermentation exhibited suboptimal cell growth, a substrate cofeeding strategy with small fraction of continuously glucose doping effectively addressed this limitation, which provided a solution to overcome undesirable substrate preferences. Further details regarding strategies for substrate cofeeding are provided in WO 2019/006301 (PCT/US2018/040290), the entire contents of which is hereby incorporated by reference in its entirety. The efficient acetate-to-squalene conversion in this study, on the other hand, presents the potential of acetic acid as an alternative low-cost feedstock to be upgraded biologically into a variety of value-added chemicals. Acetic acid can be generated from biological CO₂ fixation, lignocellulosic biomass degradation, and industrial waste stream digestion (47). Therefore, the usage of acetic acid as carbon source to produce biofuels and green chemicals would benefit to achieve net-zero greenhouse gas emissions. Overall, these findings provided an efficient approach for the biotechnological production of high value terpenoids.

### Modified Yeast Cells

Aspects of the present disclosure relate to a modified yeast cell. In some embodiments, the modified yeast cell is an oleaginous cell. In some embodiments, the oleaginous cells are oleaginous yeast cells that utilize acetate for cell growth and product synthesis. For example, in some embodiments the oleaginous yeast cells are Yarrowia lipolytica cells. Y. lipolytica is a non-pathogenic oleaginous yeast that can use a variety of carbon sources, including organic acids, hydrocarbons and various fats and oils. The term “oleaginous” refers to a microbe that can accumulate more than 20% of its dry cell weight as lipid (see C. Ratledge et al., Microbial routes to lipids. Biochem Soc Trans. 1989 December; 17(6):1139-41). Exemplary oleaginous cells include yeasts such as Yarrowia lipolytica, Candida 107, Rhodotorula glutinis, Rhodosporidium toruloides, Cryptococcus curvatus, Trichosporon pullulan, Lipomyces lipofer, Schwanniomyces occidentalis and other species from among Yarrowia, Lipomyces, Rhodosporidium and Cryptococcus; oleaginous bacteria such as those Rhodococcus, Acinetobacter and Streptomyces; and oleaginous algae and microalgae.

Aspects of the present disclosure relate to a modified yeast cell that is capable of producing increased titers of squalene as compared to an unmodified yeast cell. Squalene is an organic triterpene with the molecular formula C₃₀H₅₀. Squalene is a colorless oil that was originally obtained from shark liver oil. The name “squalene” is derived from Squalus, a genus of sharks. Squalene is commercially useful as a vaccine adjuvant and as an ingredient in hair and topical skin products.

Squalene is a product of the mevalonate pathway. Acetyl-CoA enters the mevalonate pathway where it is converted to acetoacetyl-CoA by an acetoacetyl-CoA thiolase (e.g., ERG10). Acetoacetyl-CoA is converted to HMG-CoA by a 3-hydroxy-3-methylglutaryl-CoA synthase (e.g., ERG13), which is then converted to mevalonate by a 3-hydroxy-3-methyl-glutaryl-CoA reductase (e.g., HMGR). Mevalonate is converted to mevalonate-5-phosphate by a mevalonate kinase (e.g., ERG12), and mevalonate-5-phosphate is converted to mevalonate pyrophosphate by a phosphomevalonate kinase (e.g., ERG8). Mevalonate pyrophosphate is converted to both isopentenyl pyrophosphate (IPP) and dimethylallyl pyrophosphate (DMAPP) by an isopentenyl-diphosphate delta isomerase (e.g., IDI). IPP and DMAPP are the end products of the mevalonate pathway. In squalene biosynthesis, IPP and DMAPP are condensed to form geranyl pyrophosphate (GPP) by a geranyl pyrophosphate synthase (e.g., ERG20). GPP is then converted to farnesyl pyrophosphate (FPP) by a farnesyl pyrophosphate synthase (e.g., ERG20). Finally, FPP is converted to squalene by squalene synthase (e.g., ERG9). As is known in the art, ERG10, ERG13, tHMGR, mvaE, mvaS, ERG12, ERG8, ERG19, IDI, ERG20, and ERG9 are yeast-specific nomenclature for enzymes of the mevalonate pathway. The mevalonate pathway may be divided into two pathways, the upper mevalonate pathway and the lower mevalonate pathway. The upper mevalonate pathway includes the conversion of two acetyl-CoA molecules to acetoacetyl-CoA through the reduction of HMG-CoA to mevalonate. The lower mevalonate pathway includes the conversion of mevalonate to IPP and DMAPP.

The present disclosure is related, at least in part, to the production of a modified yeast cell that comprises heterologous polynucleotides encoding enzymes capable of producing squalene from acetyl-CoA. In some embodiments, a modified yeast cell comprises a first set of heterologous polynucleotides encoding a first set of enzymes of the mevalonate pathway. In some embodiments, the first set of enzymes of the mevalonate pathway comprises ERG10, ERG13, tHMGR, mvaE, mvaS, ERG12, ERG8, ERG19, IDI, ERG20, ERG9, or any combination thereof.

In some embodiments, squalene production is improved by increased production of triacylglycerol. In some embodiments, a modified yeast cell comprises a heterologous polynucleotide encoding a lipase. In some embodiments, the lipase is a triacylglycerol lipase derived from Thermomyces lanuginosus. In some embodiments, a modified yeast cell comprises a second set of heterologous polynucleotides encoding a second set of enzymes having triacylglycerol synthesis activity. In some embodiments, the second set of enzymes having triacylglycerol synthesis activity comprises acetyl-CoA carboxylase (ACC1), diacylglycerol acyltransferase (DGA1), NAD⁺-dependent G3P dehydrogenase (GPD1), or any combination thereof. In some embodiments, triacylglycerol is produced from acetyl-CoA by the activity of ACC1, GPD1, and DGA1.

As described herein, the present disclosure relates to the production of squalene from acetyl-CoA. Accordingly, the present disclosure relates, at least in part, to increase the amount of available acetyl-CoA in a modified yeast cell. In some embodiments, a modified yeast cell comprises a third set of heterologous polynucleotides encoding a third set of enzymes having acetyl-CoA synthesis activity. In some embodiments, the third set of enzymes having acetyl-CoA synthesis activity comprises pyruvate carboxylase (PYC1), ATP:citrate lyase (ACL), citrate carrier YHM2, acetyl-CoA synthetase, or any combination thereof. In some embodiments, the ACL is from or derived from Mus musculus. In some embodiments, the acetyl-CoA synthetase is from or derived from Salmonella enterica. In some embodiments, the acetyl-CoA synthetase comprises an L641P amino acid substitution mutation relative to a wild-type acetyl-CoA synthetase.

Acetyl-CoA production may also be increased by increasing β-oxidation activity in a modified yeast cell. In some embodiments, a modified yeast cell comprises a fourth set of heterologous polynucleotides encoding a fourth set of enzymes having β-oxidation activity. In some embodiments, the fourth set of enzymes having β-oxidation activity comprises POX1, POX2, POX3, POX4, POX5, POX6, multifunctional β-oxidation protein (MFE1), 3-ketoacyl-CoA thiolase (POT1), or any combination thereof.

### Peroxisomal Targeting Signals

Aspects of the present disclosure relate, at least in part, to the surprising discovery that sequestering enzymes of the mevalonate pathway in the peroxisome of a modified yeast cell improves squalene production. Accordingly, the inventors of the present disclosure leveraged this discovery by labeling one or more enzymes of the mevalonate pathway with a peroxisomal targeting signal. A peroxisomal targeting signal (PTS) is an amino acid sequence that peroxisome receptors recognize and bind to. In nature, a PTS is a region of a peroxisomal protein, and it is responsible for ensuring that peroxisomal proteins are localized to the peroxisome. All peroxisomal proteins are synthesized in the cytoplasm and must be directed to the peroxisome. The peroxisome consists of a matrix that is surrounded by a specific membrane. Receptors within this membrane bind to peroxisomal targeting signals and initiate transport of the labeled protein into the peroxisome. In most cases, a peroxisomal targeting signal is a short sequence, usually three amino acids in length, at the extreme carboxy tail (C-terminus) of a protein. This short sequence serves as the PTS. The prototypic sequence is serine-lysine-leucine (-SKL). This motif and its variations are known as PTS1 and the PTS1 receptor is encoded by the PEX5 gene. A peroxisomal targeting signal may also be found on the N-terminus (PTS2), which is recognized by a protein complex consisting of the receptor PEX7 and a co-receptor.

In some embodiments, ERG10 comprises a peroxisomal targeting signal. In some embodiments, ERG13 comprises a peroxisomal targeting signal. In some embodiments, tHMGR comprises a peroxisomal targeting signal. In some embodiments, mvaE comprises a peroxisomal targeting signal. In some embodiments, mvaS comprises a peroxisomal targeting signal. In some embodiments, ERG12 comprises a peroxisomal targeting signal. In some embodiments, ERG8 comprises a peroxisomal targeting signal. In some embodiments, ERG19 comprises a peroxisomal targeting signal. In some embodiments, IDI comprises a peroxisomal targeting signal. In some embodiments, ERG20 comprises a peroxisomal targeting signal. In some embodiments, ERG9 comprises a peroxisomal targeting signal. In some embodiments, ERG20 and ERG9 each comprise a peroxisomal targeting signal. In some embodiments, ERG10, ERG13, tHMGR, mvaE, mvaS, ERG12, ERG8, ERG19, IDI, ERG20, ERG9 each comprise a peroxisomal targeting signal. In some embodiments, one enzyme of the mevalonate pathway comprises a peroxisomal targeting signal. In some embodiments, at least two enzymes of the mevalonate pathway comprise a peroxisomal targeting signal. In some embodiments, at least three enzymes of the mevalonate pathway comprise a peroxisomal targeting signal. In some embodiments, at least four enzymes of the mevalonate pathway comprise a peroxisomal targeting signal. In some embodiments, at least five enzymes of the mevalonate pathway comprise a peroxisomal targeting signal. In some embodiments, at least six enzymes of the mevalonate pathway comprise a peroxisomal targeting signal. In some embodiments, at least seven enzymes of the mevalonate pathway comprise a peroxisomal targeting signal. In some embodiments, at least eight enzymes of the mevalonate pathway comprise a peroxisomal targeting signal. In some embodiments, at least nine enzymes of the mevalonate pathway comprise a peroxisomal targeting signal.

In some embodiments, each enzyme of the first set of enzymes of the mevalonate pathway comprises a peroxisomal targeting signal. In some embodiments, ERG20 and ERG9 each comprise a peroxisomal targeting signal. In some embodiments, the peroxisomal targeting signal is at the C-terminus of the one or more of the first set of enzymes of the mevalonate pathway. In some embodiments, the peroxisomal targeting signal is at the N-terminus of the one or more of the first set of enzymes of the mevalonate pathway. In some embodiments, the peroxisomal targeting signal is peroxisomal targeting sequence 1 (PTS1), peroxisomal targeting sequence 2 (PTS2), or peroxisomal targeting sequence 3 (PTS3). In some embodiments, the PTS1 is the amino acid sequence serine-lysine-leucine (SKL).

### Methods

Aspects of the present disclosure relate to a method of producing squalene, comprising: culturing a modified yeast cell as disclosed herein in a culture medium for a sufficient time to produce squalene in the peroxisome of the modified yeast cell.

In some embodiments, the method further comprises extracting squalene from the modified yeast cell culture. In some embodiments, the culture medium comprises yeast extract, peptone, glucose, and an aqueous buffer. In some embodiments, the culture medium comprises a carbon substrate. The term “carbon substrate” is known in the art and refers to a carbon-based component that is contained in the culture medium, and that supplies the cultured organism with carbon and energy to perform varied cellular functions. In some embodiments, the carbon substrate is any sugar substrate that is consumed by the cell. For example, in some embodiments, the carbon substrate is any one of the following sugars: glucose, fructose, galactose, xylose, gluconate, glycerol, or other hexose sugars (e.g., allose, altrose, gulose, idose, talose, psicose, tagatose, and sorbose). In some embodiments, the culture medium comprises acetate. In some embodiments, the culture medium comprises acetate and a limited amount of one or more sugars. In some embodiments, the one or more sugars comprise glucose, fructose, and/or xylose. In some embodiments, the culture medium comprises a buffer, optionally wherein the buffer is phosphate-buffered saline.

In some embodiments, the method comprises culturing a modified yeast cell in a culture vessel. A culture vessel may be alternately referred to as a fermentor and may contain either aerobic or anaerobic conditions and a fermentation medium to suit the needed environments of the organisms in culture. The term “fermentor” or “culture vessel” refers to an enclosure, or partial enclosure, in which a biological and/or chemical reaction takes place, at least part of which involves a living organism or part of a living organism. Where liquid cultures are used for fermentation, the fermentor is typically a culture vessel able to hold the desired amount of liquid media. If a gaseous phase is employed in the fermentation process, the fermentor employed will have a volume allowing accommodation of the gaseous phase and, if the gaseous phase is not air, the fermentor is typically sealed in an airtight manner. Typically, a fermentor comprises one or more inflows and/or outflows for the introduction and/or removal of liquids, solids, and/or gas into and/or out of the fermentor.

Suitable fermentor configurations will be apparent to those of skill in the art. For example, in some embodiments, a continuous stirred tank reactor (CSTR), a bubble column reactor (BCR), a semi-batch reactor, or a trickle bed reactor (TBR), may be employed. In some embodiments, a fermentor comprises a culture of microbial cells performing the fermentation process. In some embodiments, a fermentor may continuously or semi-continuously be fed with new microbes from a growth or culture vessel.

Depending on the fermentation scale, fermentors can range from volumes of milliliters to thousands of liters or more. Some fermentors may include cell cultures where microbes (also referred to as microbial cells herein) are in contact with moving liquids and/or gas bubbles. Microbes or microbe cultures may be grown in suspension or attached to solid phase carriers. Non-limiting examples of carrier systems include microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. Carriers can be fabricated from materials such as dextran, gelatin, glass, and cellulose.

The term “aerobic conditions” is art recognized and refers to conditions that provide sufficient oxygen for efficient oxidation of a carbon source by an aerobic organism. In some embodiments, aerobic conditions are conditions that provide an abundance or even an overabundance of oxygen, for example, in the form of micro-bubbles of oxygen in a liquid medium. For example, a fermentor comprising a gaseous phase comprising at least 10%, at least 15%, at least 20%, at least 30%, at least 50%, or more oxygen is referred to as an aerobic fermentor.

The term “anaerobic conditions” is art recognized and refers to conditions that do not provide sufficient oxygen for efficient carbon oxidation by an aerobic organism. In some embodiments, anaerobic conditions are characterized by the essential absence of oxygen. In other embodiments, the oxygen content is less than required by a microbe employed to efficiently oxidize a carbon source. For example, a fermentor comprising a liquid medium and a gaseous phase comprising less than 5%, less than 2%, less than 1%, less than 0.5%, less than 0.1%, less than 0.01%, or less than 0.001% oxygen is referred to as an anaerobic fermentor.

The term “culturing” refers to maintaining a culture of an organism, for example, a microbe described herein, for a period of time, generally, for a period of time sufficient for a desired fermentation process to be carried out by the microbe. In some embodiments, the culture comprises a microbe described herein and a medium, for example, a liquid medium.

In some embodiments, the culture comprises a carbon source, for example a carbon source dissolved in the culture medium. For example, in some embodiments, a microbe is cultured in an aerobic fermentor in a liquid medium in the presence of a carbon source (e.g., acetate, or a soluble sugar) dissolved in the medium. In some embodiments, the culture comprises a salt and/or buffer establishing conditions of salinity, osmolarity, and pH, that are amenable to survival, growth, and/or conversion of the carbon source to a biofuel or biofuel precursor by the cultured organism.

In some embodiments, the culture comprises one or more additional components, for example, an additive. Non-limiting examples of additives are nutrients, enzymes, amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethylsulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise a compound, for example, a small molecule compound or drug, inducing or inhibiting transcription from a conditional or inducible promoter, for example doxicycline, tetracycline, tamoxifen, IPTG, hormones, or metal ions.

While the specific culture conditions, for example, the concentration of the carbon source, will depend upon the respective microorganism to be cultured, general methods and culture conditions for the generation of microbial cultures are well known to those of skill in the art, and are described, for example, in J. Sambrook and D. Russell, Molecular Cloning: A Laboratory Manual, Cold Spring Harbor Laboratory Press; 3rd edition (Jan. 15, 2001); David C. Amberg, Daniel J. Burke; and Jeffrey N. Strathern, Methods in Yeast Genetics: A Cold Spring Harbor Laboratory Course Manual, Cold Spring Harbor Laboratory Press (April 2005); John N. Abelson, Melvin I. Simon, Christine Guthrie, and Gerald R. Fink, Guide to Yeast Genetics and Molecular Biology, Part A, Volume 194 (Methods in Enzymology Series, 194), Academic Press (Mar. 11, 2004); Christine Guthrie and Gerald R. Fink, Guide to Yeast Genetics and Molecular and Cell Biology, Part B, Volume 350 (Methods in Enzymology, Vol 350), Academic Press; 1st edition (Jul. 2, 2002); and Christine Guthrie and Gerald R. Fink, Guide to Yeast Genetics and Molecular and Cell Biology, Part C, Volume 351, Academic Press; 1st edition (Jul. 9, 2002), all of which are incorporated by reference herein.

In some embodiments, a modified yeast cell is cultured for a sufficient time for the modified yeast cell to convert acetyl-CoA to squalene. In some embodiments, the sufficient time is at least 6 hours, at least 12 hours, at least 24 hours, at least 48 hours, at least 72 hours, at least 96 hours, or at least 192 hours.

In some embodiments, at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is in the culture medium. In some embodiments, at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is extracted from the modified yeast cell culture. In some embodiments, at least at least 25 mg/g dry cell weight (DCW), at least 50 mg/g DCW, at least 100 mg/g DCW, at least 150 mg/g DCW, or more than 150 mg/g DCW of squalene is extracted from the modified yeast cell culture.

### Additional Embodiments

1. A method of producing squalene in a yeast, the method comprising
   providing an oleaginous yeast;
   introducing modifications in the oleaginous yeast to elevate squalene production, wherein the modifications comprise
      introducing genes encoding the mevalonate pathway and its downstream squalene synthesis pathway into the peroxisome of the yeast;
      introducing a gene encoding the heterologous lipase from Thermomyces lanuginosus (tlTGL); and
      overexpressing genes encoding enzymes responsible for triacylglycerol synthesis;
   growing the modified yeast in a medium with a carbon source; and
   harvesting the squalene from the peroxisomes of the modified yeast.
2. The method of embodiment 1, wherein the modifications introduced into the oleaginous yeast further comprise
   upregulating the ATP:citrate lyase (ACL)-based cytosolic acetyl-CoA formation pathway, and
   overexpressing pyruvate carboxylase PYC1, MmACL from Mus musculus, and mitochondrial carrier YHM2.
3. The method of any one of embodiments 1-2, wherein the oleaginous yeast is Yarrowia lipolytica.
4. The method of any one of embodiments 1-3, wherein the medium comprises a yeast extract, peptone, glucose and an aqueous buffer.
5. The method of any one of embodiments 1-4, wherein the medium comprises acetate.
6. The method of any one of embodiments 1-5, wherein squalene production was more than 0.5 g/L, more than 1.0 g/L, or more than 1.5 g/L.
7. The method of any one of embodiments 1-6, wherein squalene production was more than 50 mg/g DCW, more than 100 mg/g DCW, or more than 150 mg/g DCW.
8. The method of any one of embodiments 1-7, wherein the growing the modified yeast in a medium with a carbon source comprises cultivating the cells for at least 24 hours, at least 48 hours; at least 72 hours, at least 96 hours, or at least 192 hours.
9. An oleaginous yeast comprising the modifications of embodiment 1.
10. An oleaginous yeast comprising the modifications of embodiment 2.
11. An oleaginous yeast comprising the modifications of any one of embodiments 1-2 wherein the oleaginous yeast is Yarrowia lipolytica.
12. A modified yeast cell, comprising:
   a first set of heterologous polynucleotides encoding a first set of enzymes of the mevalonate pathway, wherein one or more of the first set of enzymes of the mevalonate pathway is/are linked to a peroxisomal targeting signal;
   a first heterologous polynucleotide encoding a lipase; and
   a second set of heterologous polynucleotides encoding a second set of enzymes having triacylglycerol synthesis activity.
13. The modified yeast cell of embodiment 12, further comprising a third set of heterologous polynucleotides encoding a third set of enzymes having acetyl-CoA synthesis activity.
14. The modified yeast cell of embodiment 12 or 13, further comprising a fourth set of heterologous polynucleotides encoding a fourth set of enzymes having β-oxidation activity.
15. The modified yeast cell of any one of embodiments 12-14, wherein the first set of enzymes of the mevalonate pathway comprises ERG10, ERG13, tHMGR, mvaE, mvaS, ERG12, ERG8, ERG19, IDI, ERG20, ERG9, or any combination thereof.
16. The modified yeast cell of any one of embodiments 12-15, wherein each enzyme of the first set of enzymes of the mevalonate pathway comprises a peroxisomal targeting signal.
17. The modified yeast cell of any one of embodiments 12-15, wherein ERG20 and ERG9 each comprise a peroxisomal targeting signal.
18. The modified yeast cell of any one of embodiments 12-17, wherein the peroxisomal targeting signal is at the C-terminus of the one or more of the first set of enzymes of the mevalonate pathway.
19. The modified yeast cell of any one of embodiments 12-17, wherein the peroxisomal targeting signal is at the N-terminus of the one or more of the first set of enzymes of the mevalonate pathway.
20. The modified yeast cell of any one of embodiments 12-19, wherein the peroxisomal targeting signal is peroxisomal targeting sequence 1 (PTS1), peroxisomal targeting sequence 2 (PTS2), or peroxisomal targeting sequence 3 (PTS3).
21. The modified yeast cell of embodiment 20, wherein the PTS1 is the amino acid sequence serine-lysine-leucine (SKL).
22. The modified yeast cell of any one of embodiments 12-21, wherein the lipase is a triacylglycerol lipase derived from Thermomyces lanuginosus.
23. The modified yeast cell of any one of embodiments 12-22, wherein the second set of enzymes having triacylglycerol synthesis activity comprises acetyl-CoA carboxylase (ACC1), diacylglycerol acyltransferase (DGA1), NAD⁺-dependent G3P dehydrogenase (GPD1), or any combination thereof.
24. The modified yeast cell of any one of embodiments 13-23, wherein the third set of enzymes having acetyl-CoA synthesis activity comprises pyruvate carboxylase (PYC1), ATP:citrate lyase (ACL), citrate carrier YHM2, acetyl-CoA synthetase, or any combination thereof.
25. The modified yeast cell of embodiment 24, wherein the ACL is derived from Mus musculus.
26. The modified yeast cell of embodiment 24 or 25, wherein the acetyl-CoA synthetase is derived from Salmonella enterica.
27. The modified yeast cell of embodiment 26, wherein the acetyl-CoA synthetase comprises an L641P amino acid substitution mutation relative to a wild-type acetyl-CoA synthetase.
28. The modified yeast cell of any one of embodiments 14-27, wherein the fourth set of enzymes having β-oxidation activity comprises POX1, POX2, POX3, POX4, POX5, POX6, multifunctional β-oxidation protein (MFE1), 3-ketoacyl-CoA thiolase (POT1), or any combination thereof.
29. The modified yeast cell of any one of embodiments 12-28, wherein the modified yeast cell is an oleaginous yeast cell.
30. The modified yeast cell of embodiment 29, wherein the oleaginous yeast cell is a Yarrowia cell.
31. The modified yeast cell of embodiment 30, wherein the Yarrowia cell is Yarrowia lipolytica.
32. A method of producing squalene, comprising: culturing a modified yeast cell according to any one of embodiments 12-31 in a culture medium for a sufficient time to produce squalene in the peroxisome of the modified yeast cell.
33. The method of embodiment 32, further comprising extracting squalene from the modified yeast cell culture.
34. The method of embodiment 32 or 33, wherein the culture medium comprises yeast extract, peptone, glucose, and an aqueous buffer.
35. The method of any one of embodiments 32-34, wherein the culture medium comprises acetate.
36. The method of any one of embodiments 32, 33, and 35, wherein the culture medium comprises acetate and a limited amount of one or more sugars.
37. The method of embodiment 36, wherein the one or more sugars comprise glucose, fructose, and/or xylose.
38. The method of any one of embodiments 32-37, wherein the culture medium comprises a buffer, optionally wherein the buffer is phosphate-buffered saline.
39. The method of any one of embodiments 32-38, wherein the sufficient time is at least 6 hours, at least 12 hours, at least 24 hours, at least 48 hours, at least 72 hours, at least 96 hours, or at least 192 hours.
40. The method of any one of embodiments 32-39, wherein at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is in the culture medium.
41. The method of any one of embodiments 32-40, wherein at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is extracted from the modified yeast cell culture.
42. The method of any one of embodiments 32-41, wherein at least at least 25 mg/g dry cell weight (DCW), at least 50 mg/g DCW, at least 100 mg/g DCW, at least 150 mg/g DCW, or more than 150 mg/g DCW of squalene is extracted from the modified yeast cell culture.

### EXAMPLES

### Example 1: Revealing Rate-Limiting Steps in the Squalene Biosynthetic Pathway

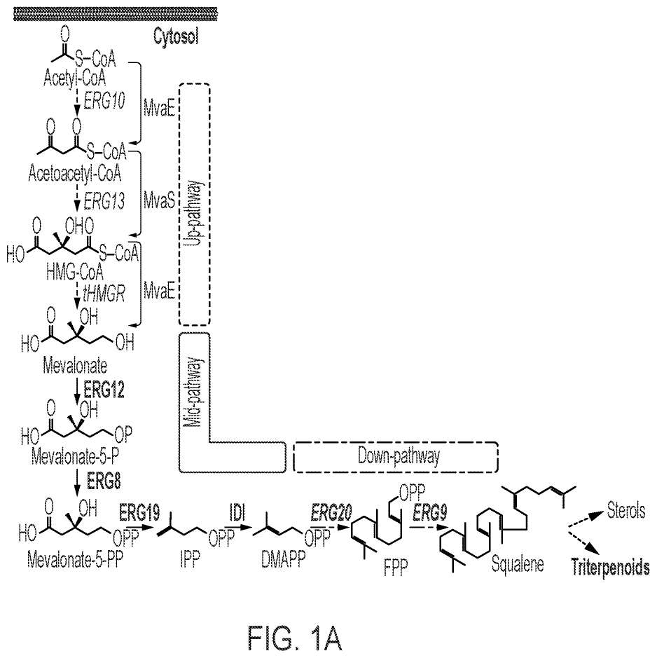

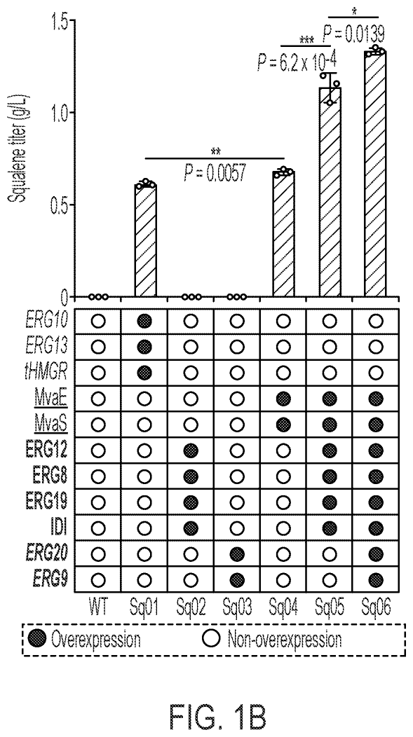

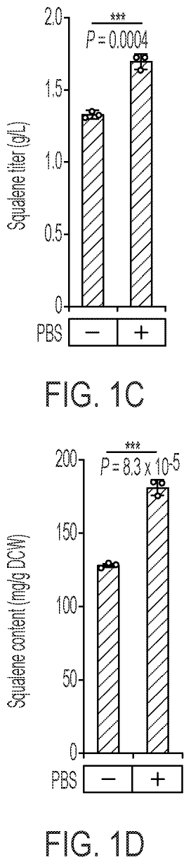

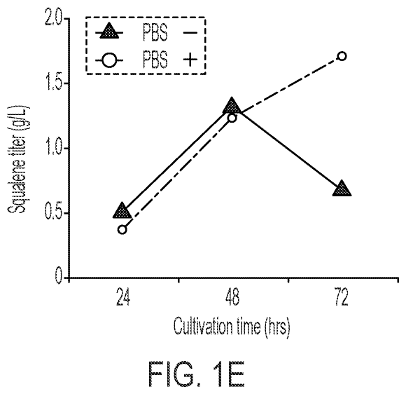

In yeast, squalene is only synthesized as an intermediate in the sterol biosynthetic pathway (FIG. 1A), which is essential for cell growth and viability. The cytosolic farnesyl pyrophosphate synthetase (ERG20) functions as a bifunctional synthase that sequentially catalyzes the condensation of isopentenyl diphosphate (IPP) and dimethylallyl pyrophosphate (DMAPP), which generates through the mevalonate (MVA) pathway, to form farnesyl diphosphate (FPP). The latter is subsequently converted by ERG9, encoding squalene synthase, to synthesize squalene (FIG. 1A). Due to the tightly regulated and balanced sterol synthesis pathway formed throughout evolution, the content of native synthesized squalene in wild-type Y. lipolytica Polf is extremely low, only 1.8 mg/L (FIG. 1B). To construct the cell factories for squalene overproduction, it was first set out to determine possible rate-limiting steps in squalene biosynthetic pathway, which would identify targets for further optimization. To this end, the squalene biosynthetic pathway started from acetyl-CoA was divided into three modules: up-pathway (MVA upstream containing ERG10, ERG13, and tHMGR), mid-pathway (MVA downstream containing ERG12, ERG8, ERG19, and IDI), and down-pathway (squalene forming route containing ERG20 and ERG9) (FIG. 1A). In addition, the capacity of the heterologous Enterococcus faecalis MvaE and MvaS proteins that catalyze the first three steps of the MVA pathway (corresponding to the steps catalyzed by ERG10, ERG13, and tHMGR proteins) (FIG. 1A) were investigated, owing to their good performance in S. cerevisiae (27, 28).

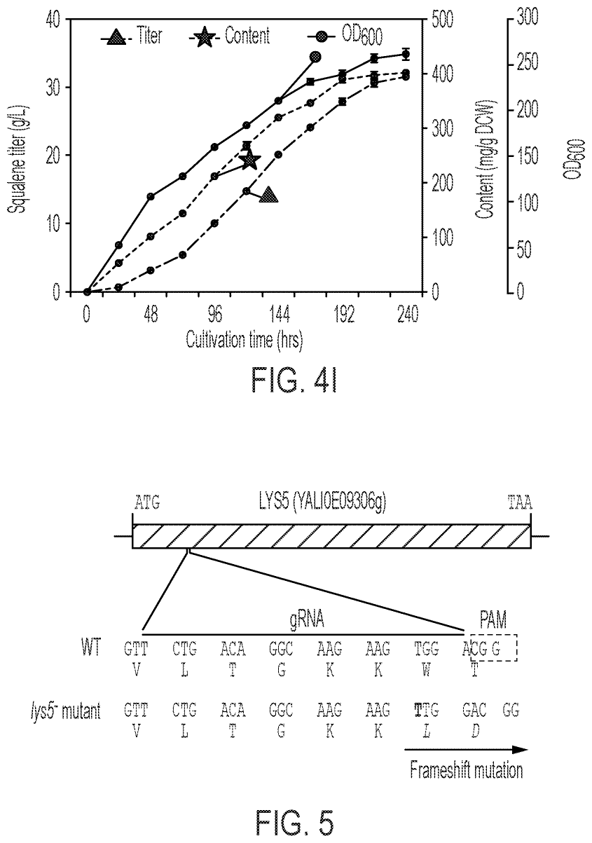

Each module was respectively overexpressed in Y. lipolytica Polf-T-L strain, a variant PoIf with TRP1 and LYS5 disruption (29) (FIG. 5), and their capacity for squalene production were assessed. Up-regulation of up-pathway revealed that the enzymes ERG10, ERG13, and tHMGR are main bottlenecks in the pathway, as their combined overexpression led to 344-fold increase (a titer of 0.62 g/L) in squalene production relative to its parent strain (Sq01 vs WT, FIG. 1B). In contrast, the effect of mid- or down-pathway overexpression on squalene production was very limited (only 1.2 or 1.3-fold increase over the parent strain, Sq02 or Sq03 vs WT, FIG. 1B), suggesting that the corresponding enzymes do not catalyze a limiting step. Intriguingly, strain harboring heterologous MvaE and MvaS produced a titer of 0.69 g/L squalene, and significantly increased by 11.3% in squalene production compared to that of overexpressing endogenous up-pathway enzymes (ERG10, ERG13, and tHMGR) (Sq04 vs Sq01, FIG. 1B). Thus, the MvaE and MvaS pair as alternative shortcut for up-pathway optimization were used for all further studies. Unexpectedly, when the expression levels of both up- and mid-pathway were up-regulated together or an entire squalene synthetic pathway was overexpressed, an additional 1.7 or 2.0-fold increase in squalene production was observed, ultimately reaching a titer of 1.34 g/L in Sq06 (FIG. 1B), possibly due to the improved availability of pathway intermediates.

These results suggest that, for the most part, the up-regulation of squalene synthetic pathway in cytoplasm functions efficiently in its biosynthesis (744-fold increase in Sq06 vs WT). An important step is the up-pathway (MVA upstream route), which is involved in competing common precursor acetyl-CoA with de novo lipid formation in Y. lipolytica and maximizing its conversation to intermediate MVA. On the other hand, while individual mid- and down-pathway up-regulation barely affected squalene production, in the presence of up-pathway overexpression, their roles appear to be extremely evident. This indicates up-regulation of up-pathway provides adequate amounts of MVA pool, which, in turn, require high-level expression of the downstream enzymes to be fully harvested.

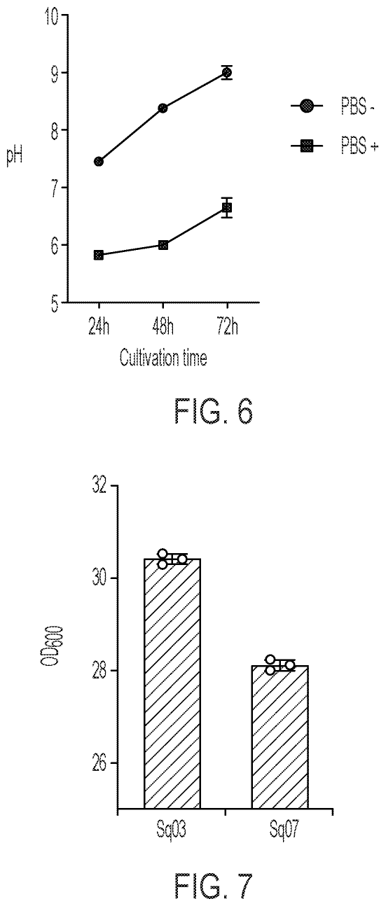

It was found that the squalene production in terms of both titer (1.71 g/L, FIG. 1C) and cellular content (182.8 mg/g DCW, FIG. 1D) was significantly increased by 27% and 41%, respectively, when YPD medium was supplemented with 0.2 M phosphoric buffer solution (PBS, pH 6.0). This possibly attributed to the pH control in the medium conditioned with PBS compared to the uncontained PBS medium (FIG. 6). The latter led to a significant pH variation which may negatively affect cell physiology, including the permeability of cell membrane and transportation process of nutrients (30). In addition, the pH-controlled fermentation process prolonged the squalene biosynthesis span and reached the highest titer at 72 hours cultivation, whereas the optimal production in the pH-uncontrolled fermentation was observed at 48 hours cultivation, then decreased significantly (FIG. 1E). Thus, the PBS-conditioned medium was used for further studies.

### Example 2: Establishing an Orthogonal Route in Peroxisome for Squalene Build-Up

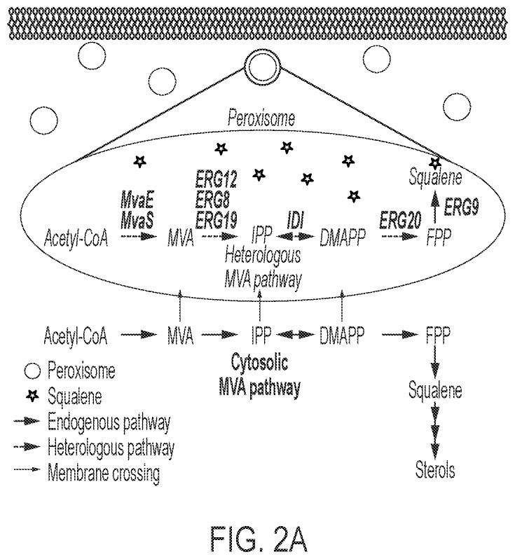

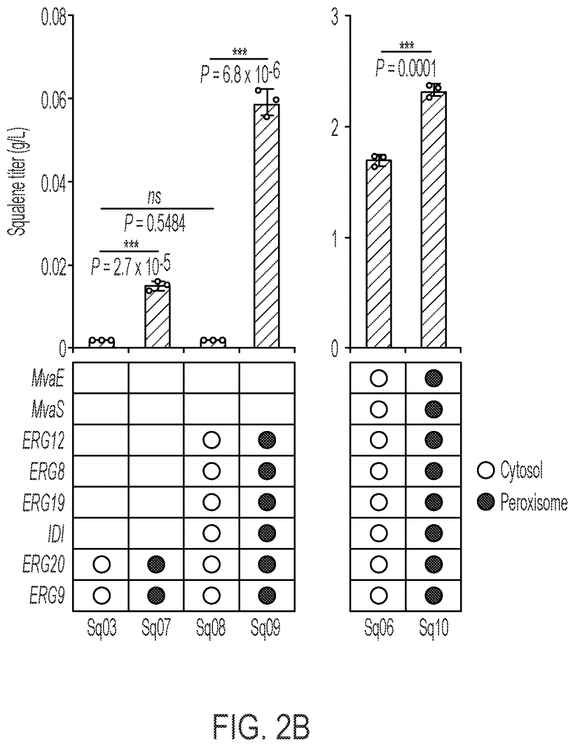

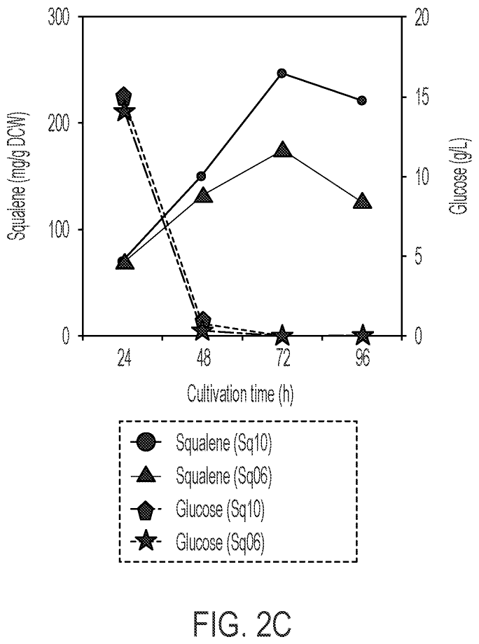

While up-regulated cytosolic squalene synthesis pathway contributed to the improvement of squalene production, the strong competition by cytosolic sterol synthesis optimized throughout the evolution makes it particularly challenge for further squalene accumulation in cytosol. Therefore, further efforts toward sequestration of squalene from its competing pathway seems to be critical and urgent to ensure sustainable build-up of squalene. Eukaryotic cells have devised solutions to overcome similar challenges by confining metabolic pathways within intracellular compartments to streamline reaction cascades and shield intermediates from competing pathways (31). In this regard, it was hypothesized that establishing an orthogonal pathway in yeast organelle for squalene synthesis would prevent squalene diversion from a strongly cytosolic competing pathway and allow a large enough squalene pool to build up. To implement this strategy, the peroxisomes were focused on, because of the following advantages: (a) peroxisomes are not essential for cell viability (32), which allows to be extensively engineered without affecting yeast fitness; (b) they are the sites where 3-oxidation of fatty acids takes place, creating a pool of acetyl-CoA that can be harvested by heterologous MVA pathway; (c) peroxisomes are also serving as storage compartments for lipophilic compounds (9), which would provide adequate space for intracellular squalene accumulation. The above-mentioned favorable characteristics of the peroxisomes provide the potential of advantageously repurposed for establishing an orthogonal metabolic network for efficient squalene synthesis (FIG. 2A).

To evaluate the suitability of the peroxisome for squalene production, it was first set out to direct the down-pathway containing ERG20 and ERG9 to the peroxisome by addition of a C-terminal SKL peroxisomal targeting signal (FIG. 2A). Squalene production drastically improved by 6.3-fold, compared to that of their cytosolic overexpression (Sq07 vs Sq03, FIG. 2B). In addition, a higher biomass in strain Sq03 was observed over Sq07 (FIG. 7), which can be possibly attribute to more squalene channeled into sterol pathway in Sq03, thereby promoting the cell growth. These results supported the initial hypothesis that the peroxisome is able to act as a barrier and insulate the locally formed squalene from cytosolic sterol synthesis. It also suggested that precursors IPP and DMAPP generated by cytosolic MVA pathway could be translocated into peroxisome from the cytosol.

To expand on these findings, the possibility to harvest the peroxisomal acetyl-CoA pool for squalene production in this organelle was assessed. To this end, the MVA pathway enzymes were sequentially introduced to the peroxisome. In the presence of ERG20 and ERG9, sequential addition of mid-pathway (ERG12, ERG8, ERG19, and IDI) in the peroxisome resulted in additional 3.8-fold increase of squalene production (Sq09 vs Sq07, FIG. 2B), while their cytosolic overexpression only had a minor impact on squalene production (Sq08 vs Sq03, FIG. 2B). When the entire MVA pathway was assembled in the peroxisome, forming a complete orthogonal route to harvest the peroxisomal acetyl-CoA, a strong additional 40-fold increase in squalene production was observed, yielding 2.34 g/L in strain Sq10 (Sq10 vs Sq09, FIG. 2B), which also showed 1.37-fold higher than that of strain Sq06 overexpressing MVA pathway in cytosol (Sq10 vs Sq06, FIG. 2B). These data suggested that the heterologous peroxisomal MVA pathway is able to harvest the available pool of peroxisomal acetyl-CoA. The establishment of the orthogonal metabolic network in peroxisome showed the potential of bypassing current limitations in squalene production imposed by the strong competition of the sterol synthesis in the yeast cytosol. This was further confirmed by the time course of squalene concentration (FIG. 2C). At the initial phase (24 h), the strain Sq06 had a similar squalene production rate to the peroxisome pathway containing strain Sq10 (FIG. 2C). However, after that, especially glucose depletion (48-72 h), the squalene productivity in strain Sq06 gradually decreased (FIG. 2C), possibly formed squalene in cytosol channeled into sterol pathway, whereas the strain Sq10 still kept an elevated productivity for squalene build-up in peroxisome (FIG. 2C).

To note, despite the 37% increase squalene production in strain Sq10 over Sq06, it seems to be slight increase, if comparing it to the fold change between Sq07 and Sq03 (6.3-fold) or Sq09 and Sq08 (24.4-fold) (FIG. 2B). This suggested that insufficient acetyl-CoA supply in peroxisome possibly compromised the performance of the peroxisomal pathway, thus creating a major bottleneck for further squalene accumulation in peroxisome. Conversely, adequate acetyl-CoA pool in cytosol may compensate for the loss of squalene into sterol pathway, as a result, reducing the gap of squalene production between cytosolic engineering strain Sq06 and peroxisome engineering strain Sq10 (FIG. 2B).

### Example 3: Transforming by-Product Lipid into Peroxisomal Squalene Synthesis

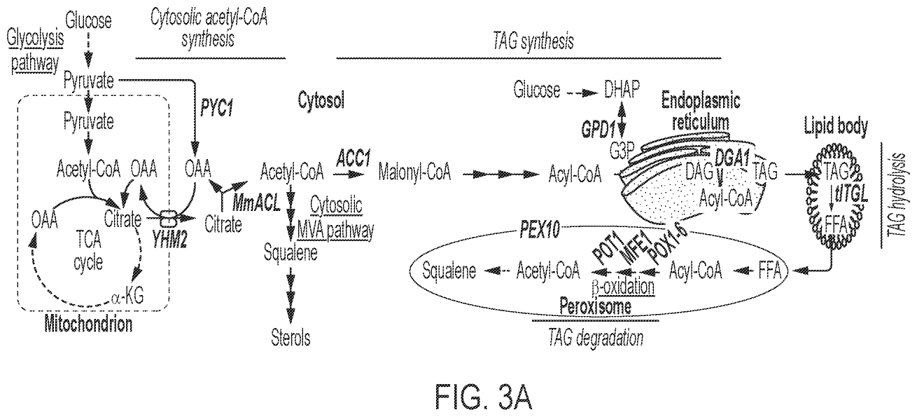

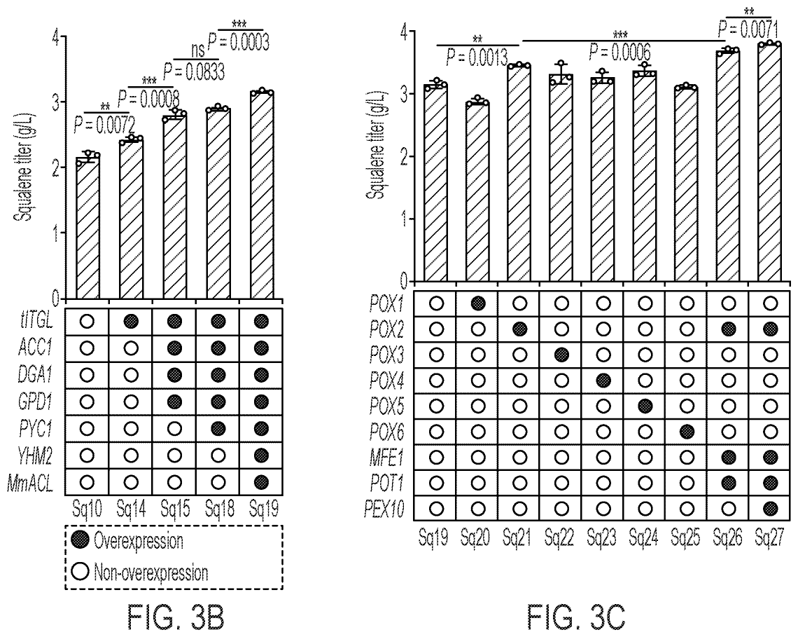

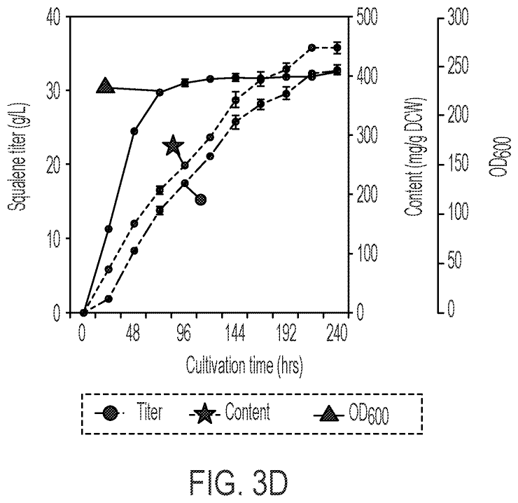

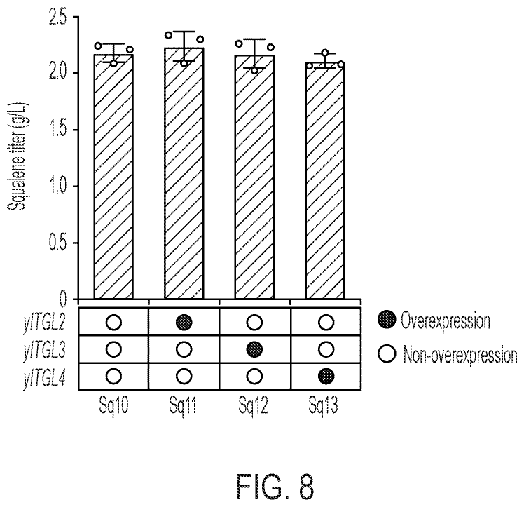

Next, the enhancement of precursor acetyl-CoA supply in peroxisome was focused on through promoting lipid metabolism including its biosynthesis, hydrolysis and degradation processes (FIG. 3A). Y. lipolytica is a natural lipid producer capable of accumulating 30%-60% dry cell weight (30), mainly in the form of triacylglycerols (TAGs) stored in lipid body. Although lipid body in Y. lipolytica generally facilitate lipophilic compound sequestration and storage, engineered peroxisome would serve as a similar storage compartment for locally formed squalene accumulation, which in turn makes lipid body unnecessary for target storage. It was hypothesized that the accumulated TAGs in lipid body would serve as carbon source to support peroxisomal squalene production. To test this notion, it was attempted to convert the intracellular TAGs to free fatty acid (FFA) that is degraded via β-oxidation to generate acetyl-CoA in peroxisome, by overexpressing either the endogenous lipases (ylTGLs) mediated TAG hydrolysis or a heterologous tlTGL from Thermomyces lanuginosus (33) in peroxisomal squalene-producing strain Sq10 (FIG. 3A). The ylTGLs overexpression slightly improved squalene production (FIG. 8), while the introduction of lipid body-targeted tlTGL significantly improved squalene titer by 12% (Sq14 vs Sq10, FIG. 3B). This result suggested that intracellular TAGs were funneled into peroxisomal squalene synthesis and contribute to high yield. Meantime, it provides inspiration that there is still considerable potential for improve the peroxisomal squalene synthesis if converting acetyl-CoA pool in cytosol into peroxisomal acetyl-CoA through lipid metabolism.

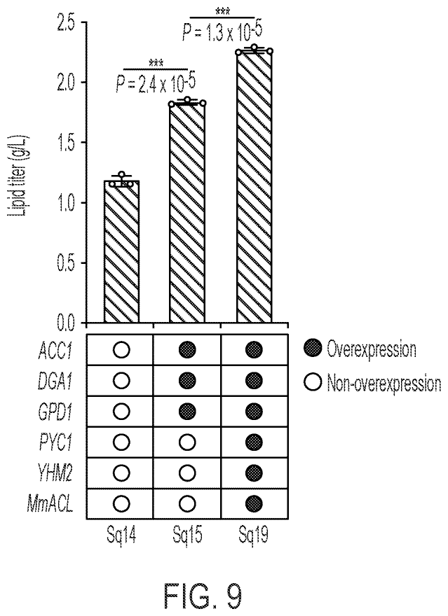

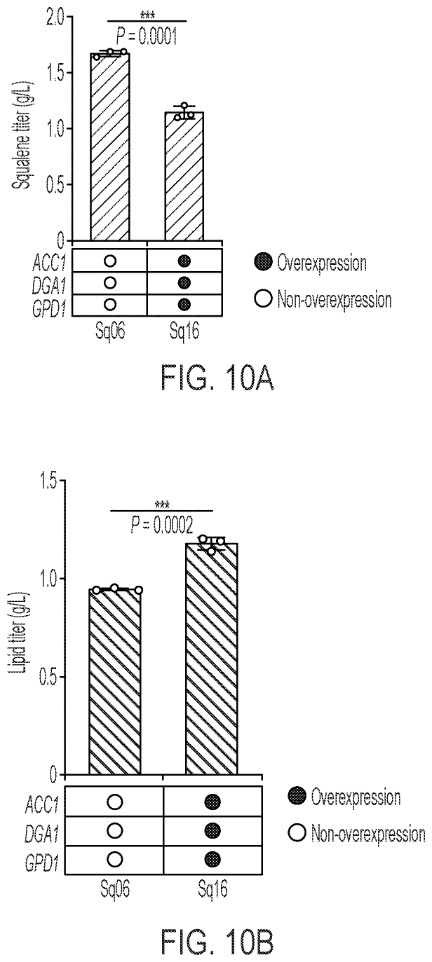

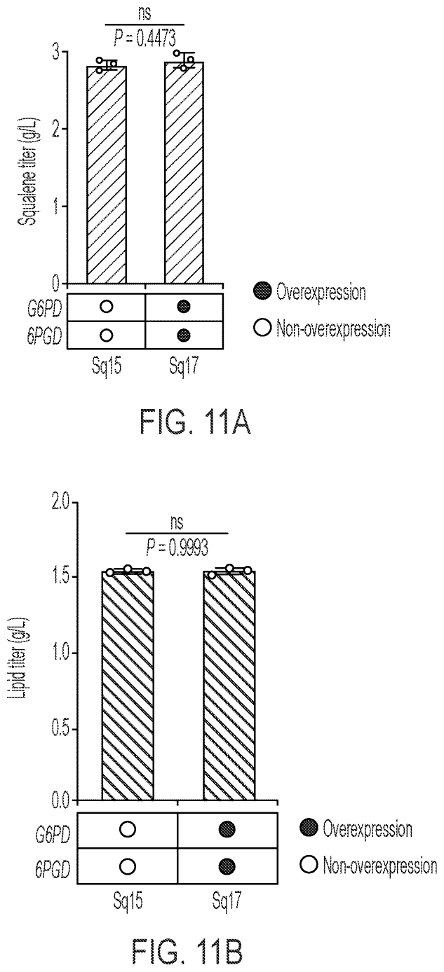

To this end, the key enzymes responsible for limiting biochemical reactions involved in TAG synthesis were overexpressed, including acetyl-CoA carboxylase (ACC1) (34), which converts acetyl-CoA into malonyl-CoA (the first committing step of TAG synthesis), diacylglycerol acyltransferase (DGA1) (34) catalyzing the ultimate step in TAG synthesis, and NAD⁺-dependent G3P dehydrogenase (GPD1) converting dihydroxyacetone phosphate (DHAP) into glycerol-3-phosphate (G3P) (35). As expected, their overexpression led to significant increase in lipid content (Sq14 vs Sq15, FIG. 9). Correspondingly, the peroxisomal squalene production has also risen considerably by 16% (Sq14 vs Sq15, FIG. 3B), which could be the result of higher precursor flux through the peroxisomal pathway. The effect on cytosolic pathway-based squalene production in strain Sq06 was investigated by overexpressing these three enzymes. A considerably lower squalene production was observed (Sq06 vs Sq16, FIG. 10A), which might be attribute to more common acetyl-CoA in cytosol channeled into lipid synthesis (FIG. 10B), and this in turn resulted in limited flux into cytosolic MVA and product-forming pathway. While lipid bodies in Y. lipolytica create hydrophobic pockets that facilitate lipophilic terpenoid sequestration and storage (36), increased TAG supply comes at the expense of common precursor acetyl-CoA in cytosol. Therefore, carbon flux needs to be optimally partitioned between lipid and terpenoid synthesis, yet it is still challenging. Alternatively, here construction of orthogonal terpenoid pathway in the peroxisome efficiently circumvented this limitation. There was also an attempt to increase cofactor NADPH supply required for de novo lipid biosynthesis (37), by overexpressing two genes G6PD (encoding glucose-6-phosphate dehydrogenase) and 6PGD (encoding phosphor-gluconate dehydrogenase) involved in pentose phosphate pathway. However, neither squalene nor lipid production improved significantly (FIG. 11A), suggesting NADH recycling not the rate-limiting steps in this case; as such, this strategy was not further pursued.

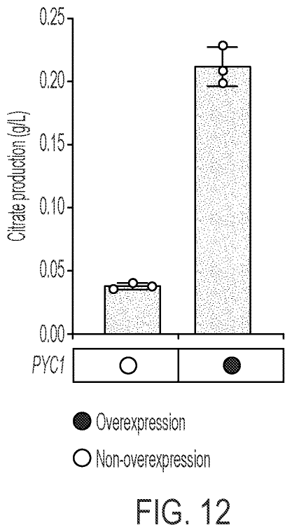

Acetyl-CoA in cytosol is the main precursor for lipid synthesis, and sufficient metabolic flux of cytosolic acetyl-CoA is critical for lipid production. The supply of cytosolic acetyl-CoA was attempted to be enhanced. Pyruvate carboxylase (PYC1) was first overexpressed to ensure efficient formation of oxaloacetate required for citrate biosynthesis and transport (FIG. 3A). However, overexpressing PYC1 resulted in marginal increase in squalene production (Sq15 vs Sq18, FIG. 3B). The increased citrate pool (FIG. 12) suggested decomposition of citrate to form acetyl-CoA and oxaloacetate catalyzed by ATP:citrate lyase (ACL) possibly being a pathway bottleneck. To overcome this limitation, a heterologous ACL was introduced from Mus musculus (MmACL) because of its higher affinity for citrate (Kₘ of 0.05 mM) than endogenous ones (Kₘ of 3.6 mM) (38). In addition, the provision of cytosolic acetyl-CoA this way also relies on mitochondrial activity for citrate exportation. Therefore, MmACL was overexpressed together with YHM2, the Y. lipolytica mitochondrial citrate carrier that was characterized as an antiporter for citrate and α-ketoglutarate or oxaloacetate (39). This led to significant increase by 8.6% in squalene production, reaching 3.17 g/L in strain Sq19 (Sq19 vs Sq18, FIG. 3B).

Although the above optimized lipid metabolic pathway potentially redirected carbon flux to peroxisome and thereby significantly promoted peroxisomal squalene synthesis (1.45-fold increase in Sq19 vs Sq10, FIG. 3B), the accumulated lipid pool in strain Sq19 (FIG. 9) suggested lipid degradation into acetyl-CoA in peroxisome possibly being the limitation for carbon-utilizing efficiency into desired compound. Therefore, it was next attempted to increase lipid-to-squalene yield mainly through strengthening β-oxidation pathway that drives acyl-CoA conversation into acetyl-CoA in peroxisome (FIG. 3A). To this end, the enzymes involved in β-oxidation process were targeted for engineering. Peroxisome β-oxidation pathway in yeast is a multistep process requiring three different enzymatic activities (FIG. 3A), including six acyl-CoA oxidases encoded by six genes POX1 to POX6 (40), which are responsible for the first committing step of β-oxidation, Multifunctional β-oxidation protein (MFE1) involving in second and third β-oxidation step (41), and 3-ketoacyl-CoA thiolase (POT1) catalyzing the last step (42). To explore the possible rate-limiting steps in β-oxidation pathway, the six acyl-CoA oxidases were first individually overexpressed in peroxisomal production strain Sq19. Of six acyl-CoA oxidases, overexpressing POX2 exhibited the most significant improvement of squalene synthesis in strain Sq21 (FIG. 3C). It should be attributed to POX2 preferentially oxidizing long-chain fatty acids (40) that are the most abundant in Y. lipolytica. Subsequently, additional co-overexpression of MFE1 and POT1 further resulted in 6.7% increase in squalene production (Sq26 vs Sq21, FIG. 3C). In addition, peroxisome population might be another key factor for peroxisomal production, because of its potential capacity of supporting more space for enzyme expression and product storage. Thus, the effect of increased peroxisome population on peroxisomal squalene production was investigated by overexpressing the peroxisome biogenesis factor 10 (PEX10) that plays a role in regulating the size and/or number of peroxisome (42). Pex10 overexpression further improved squalene production by 3.1% and yielded 3.82 g/L in strain Sq27 (Sq27 vs Sq26, FIG. 3C).

Taken together, transforming by-product lipid into peroxisomal acetyl-CoA pool led to 63% increase in squalene production in peroxisome (Sq27 vs Sq10, FIG. 3B, 3C), which suggested that engineering lipid metabolism would potentially support the synthesis of acetyl-CoA derived chemicals in peroxisome. The performance of constructed strain Sq27 in 3-L fed-batch cultivation in which glucose as carbon source was investigated. Final titers of 32.8 g/L and content of 446 mg/g DCW were obtained (FIG. 3D), with a productivity of 0.15 g/L/h. These data demonstrate the robustness of the engineering strains in large culture volumes and high-cell density fermentation.

### Example 4: Establishing an Orthogonal Acetyl-CoA Shortcut in Peroxisome

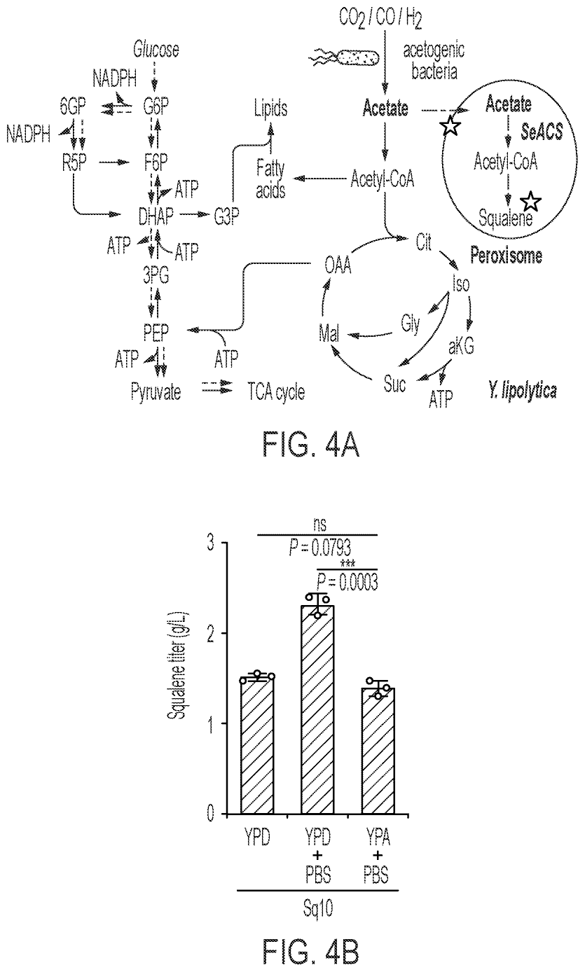

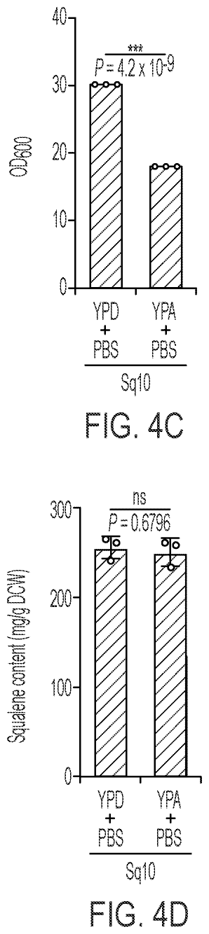

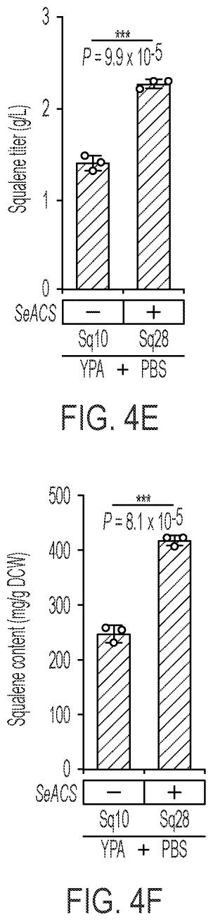

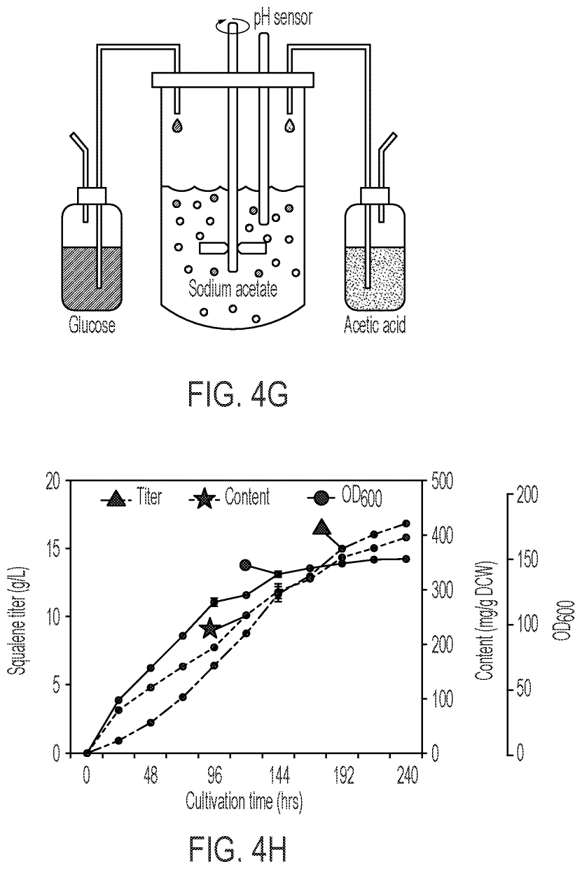

Other viable options that can enhance the supply of acetyl-CoA in peroxisome without the need to engineer such long lipid metabolic pathway mentioned above were explored. It was previously reported that Y. lipolytica possesses strong acetate utilization pathway, which would produce the core molecular acetyl-CoA directly from acetate as carbon source to support cell growth and target compound synthesis (30) (FIG. 4A). This natural capacity of Y. lipolytica can provide inspiration for solving the metabolic engineering challenges. Here it was first investigated the effect on squalene production in strain Sq10 harboring peroxisomal pathway by feeding 27.4 g/L sodium acetate as sole carbon source, equivalently to 20 g/L glucose, as well as supplementing with 0.2 M PBS (pH 6.0) to control pH in the medium. As a result, a similar or 40% decreased squalene titer was observed when the strain Sq10 was cultured in YPA medium (acetate as carbon source), compared to that of YPD medium (glucose as carbon source) supplemented with or without PBS, respectively (FIG. 4B). The decreased titer of squalene should result from the reduced cell growth when cultured in YPA medium (FIG. 4C), because similar squalene content was observed between YPD and YPA medium (FIG. 4D). This equivalent content demonstrated that Y. lipolytica could efficiently and potentially uptake acetate as sole carbon source to produce squalene.

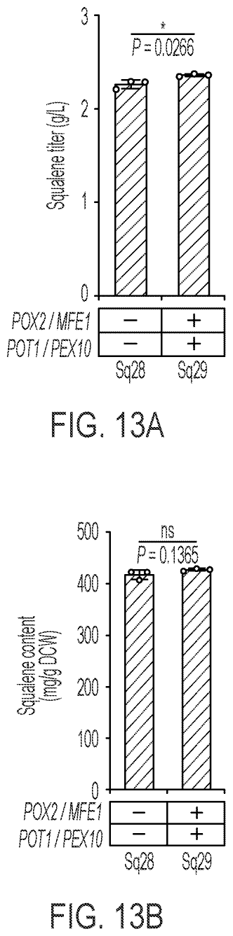

Considering the nearly perfect performance of acetate as carbon source for squalene synthesis by harnessing the endogenous cytosolic acetate uptake pathway, it was hypothesized that establishment of the orthogonal acetate utilization pathway in peroxisome could potentially improve the peroxisomal concentration of acetyl-CoA and enhance acetate-to-squalene conversion. To this end, a heterologous acetyl-CoA synthetase variant (L641P, feedback inhibition-insensitive) from Salmonella enterica (SeACS^{L641P}) (43) was introduced and targeted to the peroxisome, which in turn created a metabolic shortcut to acetyl-CoA from acetate in peroxisome for squalene synthesis (FIG. 4A). As a result, a significant increased titer of squalene by 63% was obtained (Sq28 vs Sq10, FIG. 4E) as well as its elevated cellular content by 69% (Sq28 vs Sq10, FIG. 4F), yielding 2.3 g/L and 420 mg/g DCW, respectively. To be noted, this orthogonal acetyl-CoA shortcut enabled strain Sq28 to produce the similar titer of squalene in YPA medium, if compared to that of Sq10 in YPD medium (Sq28 vs Sq10, FIGS. 4B and 4E). It was observed that the cellular squalene content in strain Sq28 reached equivalent capacity to prior strain Sq27 (obtained by first approach) grown in YPD medium (420 mg/g DCW in Sq28 vs 424 mg/g DCW in Sq27). These results strongly indicated that harnessing the orthogonal acetate utilization pathway in peroxisome could bypass the long lipid metabolic steps and created a peroxisomal acetyl-CoA shortcut to achieve metabolic optimality in producing squalene in peroxisome. In addition, POX2, MFE1, POT1 and PEX10 were also overexpressed with goals of promoting lipid degradation to acetyl-CoA. However, only slight increase of squalene production was observed in terms of both titer and cellular content (Sq28 vs Sq29, FIGS. 13A-13B), possibly adequate acetyl-CoA derived from acetate in peroxisome satisfied the downstream pathway for squalene synthesis. Next, the capacity of engineered strain Sq28 for converting acetate into squalene in a scale-up bioreactor was investigated. During the fed-batch fermentations in bioreactor, a joint feed strategy was used, in which acetate was fed in both the salt and acid forms (FIG. 4G). The latter form functions not only as carbon source but also as acid to regulate pH in medium for optimal cell growth. Eventually, squalene production with a titer of 16.8 g/L as well as cellular content of 394 mg/g DCW (FIG. 4H) was achieved.

Compared to glucose fermentation, it was found that the decreased biomass in acetate fermentation was the main bottleneck for squalene production with high titers. Therefore, further efforts towards optimal cell growth seems to be critical for acetate fermentation. Substrate mixtures provide the potential to alleviate such limitations in reductive metabolism without genetic engineering. Each substrate has unique efficiency for carbon, energy and cofactor generation, allowing fine-tuning of carbon-to-energy-to-cofactor ratios (44). Nevertheless, mixed substrate metabolism is frequently hampered by catabolite repression based on substrate preference (45), owing to the evolutionary fitness of cells in nature environments. To overcome undesirable substrate preference without reducing acetate reduction, limiting quantities of glucose was continuously supplied over the course of fermentation to an acetate culture (FIG. 4G). The glucose feed rate was kept quite slow to maintain negligible concentrations (undetectable level) in the reactor. In this fed-batch set-up, cells simultaneously consumed acetate and the supplemented glucose, with acetate remaining as the primary carbon source. Under these conditions, acetate utilization metabolism dominated to provide precursors for squalene production in peroxisome, glucose primary provided ATP sufficient and NADPH generation for cell maintenance and growth. Using this approach, nearly doubled titers of squalene (31.6 g/L, FIG. 4I) were obtained, compared to acetate-only fermentation (16.8 g/L, FIG. 4H), while a similar cellular content of squalene (402 mg/g DCW in cofeeding fermentation vs 394 mg/g DCW in acetate-only fermentation, FIGS. 4H and I). In particular, the rate of cell growth with substrate cofeeding was nearly twice as fast as that of the acetate-only control (0.327 DCW/h vs 0.178 DCW/h). In addition, the rate of acetate uptake was enhanced by 25% after glucose doping, suggesting controlled continuous feeding of a preferred substrate did not inhibit the consumption of the less favored substrate. These results demonstrated that the fed-batch cofeeding significantly outperformed the acetate-only control, providing an alternative approach for the synthesis of terpenoids or even acetyl-CoA derived chemicals sourced from acetic acid.

### Example 5: Methods and Materials Related to Examples 1-4

Culture conditions and media. E. coli DH5α cells used for plasmid propagation were grown in Luria-Bertani (LB) media (BD bioscience) supplemented with corresponding antibiotics (50 g/mL kanamycin or 100 μg/mL ampicillin) at 37° C. with constant shaking for 16 h. All Yarrowia lipolytica strains were cultured at 30° C. with shaking at 230 rpm. The media used for Y. lipolytica growth and squalene production was prepared as follows. YPD media was prepared with 10 g/L yeast extract (VWR Life Science), 20 g/L peptone (VWR Life Science), and 20 g/L glucose (Sigma-Aldrich) and supplemented with 15 g/L agar (BD bioscience) for agar plates if needed. YPA broth was prepared with 10 g/L yeast extract (VWR Life Science), 20 g/L peptone (VWR Life Science), and 27.4 g/L sodium acetate (Sigma-Aldrich). Phosphoric buffer solution (PBS) with pH 6.0 was prepared with 0.2 M Na₂HPO₄ and 0.2 M NaH₂PO₄, which was used to replace water to make YPD- or YPA-PBS fermentation media. YNB media used for selecting transformed Y. lipolytica strains was prepared with 1.7 g/L yeast nitrogen base without amino acids and ammonium sulfate (YNB, BD bioscience), 20 g/L glucose, 5 g/L ammonium sulfate (VWR Life Science), 15 g/L agar (BD bioscience), and 0.77 g/L appropriate complete supplement mixture without uracil, leucine, lysine or tryptophan (Sunrise science products). For antibiotic selection, hygromycin B (250 g/mL, Sigma-Aldrich) or nourseothricin (400 g/mL, Fisher Scientific) was added to YPD media.

Construction of plasmids and strains. E. coli DH5α (purchased from NEB) was used for plasmid propagation. Y. lipolytica po1f strain stocked in lab served as the base strain, and its derivatives and plasmids used in this study are listed in Table 1. The primers (synthesized in Sigma-Aldrich) used for plasmid construction are provided in Table 2. The restriction enzyme Not1 used for linearizing plasmid was purchased from New England Biolabs (NEB). KAPA HiFi DNA polymerase with high-fidelity (KapaBiosystems) was used for gene amplification for plasmid construction. GoTaq DNA polymerase (Promega) was used for colony PCR identification. PCR fragments were purified using the ZYMO™ Fragment Recovery Kit (ZYMO™ research). IPlasmids were constructed using NEBuilder® HiFi DNA Assembly Master Mix (NEB®), and then transformed into chemically competent E. coli DH5α cells by heat shock. The constructed plasmids were extracted by the QIAprep® Spin Miniprep Kit (Qiagen), and subsequently sequenced at Quintara Bioscience. All engineered Y. lipolytica strains were achieved by transforming linearized plasmids (Not1 digestion) using the lithium-acetate method. Recombinants were verified by PCR amplification from genomic DNA. Heterologous genes MvaE (WP_002357755.1) and MvaS (WP_002357756.1) sourced from E. faecalis, and cucurbitadienol synthase CbQ (K7NBZ9.1) sourced from S. grosvenorii were codon-optimized towards Y. lipolytica and synthesized by GeneArt (Thermo Fisher Scientific).

LYS5 disruption in Y. lipolytica strain using CRISPR-Cas9. Previously, a Y. lipolytica po1f variant po1f-T was obtained, in which TRP1 gene was disrupted using CRISPR-Cas9 technology, resulting in available three auxotrophic markers (ura3⁻, leu2⁻, trp1) (29). To exploit more available auxotrophic markers, similar approach was used to disrupt LYS5 (YALIOE09306). The CRISPR-Cas9 plasmid containing gRNA (TCTGACAGGCAAGAAGTGGA) (SEQ ID NO: 78) targeting the LYS5 gene was transformed into strain po1f-T using Ura3 as the auxotrophic marker. The strain with lysine auxotrophy was obtained by selecting on YNB-Ura and YNB-Ura-lys plates. After that, the positive clones were inoculated onto YPD plates and sub-cultured three times to lose the CRISPR-Cas9 plasmid, resulting in the po1f-T-L strain (ura3⁻, leu2⁻, trp1⁻, lys5⁻).

Shake flask fermentations. A single colony of engineered strains was picked from the plate and inoculated into 2 mL YPD media followed by cultivation at 30° C. for 16 h. The overnight culture was then transferred to a 50 mL shake flask containing 10 mL YPD or YPA media with initial OD₆₀₀ of 0.1 and cultivated at 30° C. with shaking at 230 rpm for 2 or 3 days.

Bioreactor fermentations. Fed-batch fermentations were performed in a 3 L bioreactor (New Brunswick Bioflo115 system). For glucose fermentation, the initial fermentation was completed with 1 L media containing 50 g/L yeast extract, 100 g/L peptone, 100 g/L glucose, and 0.2 M PBS with pH 6.0. The seed cultures of engineered strains were prepared by inoculating appropriate strains into YPD media and growing at 30° C./230 rpm for 18 h, and then inoculated into bioreactor with starting OD₆₀₀ of 1.5-2.0. The temperature was maintained at 30° C. The dissolved oxygen was controlled at 20% with an agitation cascade of 250~800 rpm during growth phase (typically from 0 to 48 hours). Air was sparged into fermenter at 2 vvm. Once the media feeding starting, the agitation and aeration was changed and held constantly at 600 rpm and 1 vvm, respectively. The pH was maintained at 6.5 by feeding 5 M HCL. Foam was prevented by the addition of antifoam 204 (Sigma-Aldrich). The fed-batch process was initiated after 48 hours of fermentation with rich YPD media consisting of 100 g/L yeast extract, 100 g/L peptone, 500 g/L glucose and 0.2 M PBS. For acetate fermentation, the initial fermentation was completed with 1 L media containing 20 g/L yeast extract, 40 g/L peptone, 40 g/L sodium acetate, and 0.2 M PBS with pH 6.0. The seed cultures were prepared by inoculating appropriate strains into YPD media for overnight growth, and then inoculated into YPA media for cultivation of 24 h before inoculating seed culture into bioreactor. The dissolved oxygen was controlled at 20% with an agitation cascade of 250-800 rpm. Air was sparged into fermenter at 2 vvm. The fed-batch process was initiated with feeding concentrated acetic acid (Sigma-Aldrich) as carbon source. The supplemented acetic acid also acts as a pH regulator to maintain pH at 6.8. The concentrated YP media (100 g/L yeast extract and 100 g/L peptone) prepared with 0.2 M PBS was added to provide nitrogen source for cell growth. In substrate cofeeding systems, the supplemented glucose was continuously fed at a quite slow rate to maintain its concentration in the bioreactor at undetectable level. Samples were taken every 24 hours to measure OD₆₀₀, glucose or acetate concentration, and squalene titer.

Quantification of residual glucose or acetate in media. Determination of glucose or acetate in media was carried out by High-Performance Liquid Chromatography (HPLC, Agilent technologies 1260) equipped with a refractive index detector and an HPX-87H column (Bio-Rad). 1 mL samples were collected and centrifuged at 12,000 g for 1 minute, and then the supernatant was filtered by 0.2 m syringe filters prior to injection. The mobile phase consisting of 14 mM sulfuric acid was used at a flow rate of 0.7 mL/min at 50° C. 10 μL sample was injected into HPLC.

Lipid extraction and quantification. 0.5 mL cell culture was collected and centrifuged at 12,000 g for 2 minutes, and supernatant was discarded. 0.5 mL of a 0.5 M sodium hydroxide-methanol solution was mixed with cell pellets, followed by the addition of internal standards, 2 mg/mL methyl tridecanoate (Sigma-Aldrich) and 2 mg/mL glyceryl triheptadecanoate (Sigma-Aldrich) dissolved in hexane. The mixture was vortexed for 1 hour in the room temperature to allow the transesterification of lipids to fatty acid methyl esters (FAMEs) prior to adding 40 μL purified sulfuric acid (Sigma-Aldrich) for pH neutralization. The FAMEs were then extracted by adding 0.5 mL hexane followed by vortexing for 30 minutes. The upper hexane phase was collected for analysis after a centrifugation at 12,000 g for 5 minutes. Quantification of FAMEs was performed by GC-FID (Agilent technologies) equipped with an Agilent HP-INNOWAX capillary column. The injection volume was 1 μL, split ratio was 50:1, and the inlet temperature was 260° C. The GC oven temperature was as follows: initial temperature was set 100° C., then ramped to 240° C. at a rate of 50° C./min, held for 10 minutes. Fatty acids were identified and quantified by comparison with commercial FAME standards (Sigma-Aldrich). Total lipid content was calculated as the sum of total fatty acid content for five FAMEs: methyl palmitate (C16:0), methyl palmitoleate (C16:1), methyl stearate (C18:0), methyl oleate (C18:1), and methyl linoleate (C18:2).

Extraction and quantification of squalene. 100-500 μL cell culture was collected and centrifuged for 2 minutes at at 12,000 g. Cell pellets were suspended in 500 μL methanol with glass beads (425-600 μm, Sigma-Aldrich). The mixture was vortexed for 1 hour in the room temperature, and then followed by addition of 500 μL hexane for squalene extraction by vortexing for 30 minutes. The upper hexane phase was collected for analysis after a centrifugation at 12,000 g for 5 minutes. Quantification of squalene was performed by GC-FID (Agilent technologies) equipped with an Agilent HP-INNOWAX capillary column.

The injection volume was 1 L, split ratio was 2:1, and the inlet temperature was 260° C. The GC oven temperature was as follows: initial temperature was set 100° C. for 0.5 minutes, then ramped to 250° C. at a rate of 50° C./min, held for 8 minutes. Squalene was identified and quantified by comparison with commercial squalene standard (Sigma-Aldrich). The production of squalene was expressed as grams per liter of fermentation broth (g/L) and milligrams per gram of dry cell weight (mg/g DCW). Optical densities were measured at 600 nm with Thermo Spectronic Genesys 20 (Thermo Scientific) and used to calculate cell mass with formula of DCW=0.3×OD₆₀₀.

It should be understood that the subject matter defined in the appended claims is not necessarily limited to the specific implementations described above. The specific implementations described above are disclosed as examples only.

TABLE 1
Strains and constructs.

| Strain name | Description of strains and plasmids |
|---|---|
| Po1f | MatA, leu2-270, ura3-302, xpr2-322, axp-2 |
| Po1f-T | MatA, leu2-270, ura3-302, xpr2-322, axp-2, trp1 |
| Po1f-T-L | MatA, leu2-270, ura3-302, xpr2-322, axp-2, trp1, lys5 |
| Sq01 | Po1f-T-L, LYS5:: T_{Lip1}-ERG13-P_{EXP1} + P_{TEFin}-tHMGR-T_{XPR2} + T_{Lip1}-ERG10-1-P_{EXP1} + P_{TEFin}- |
| | ERG10-1-T_{XPR2} |
| Sq02 | Po1f-T-L, LYS5:: T_{Lip1}-ERG12-P_{EXP1} + P_{TEFin}-ERG8-T_{XPR2} + T_{Lip1}-ERG19-P_{EXP1} + P_{TEFin}-IDI- |
| | T_{XPR2} |
| Sq03 | Po1f-T-L, LYS5:: T_{Lip1}-ERG20-P_{EXP1} + P_{TEFin}-ERG9-T_{XPR2} |
| Sq04 | Po1f-T-L, LYS5:: T_{Lip1}-MvaS-P_{EXP1} + P_{TEFin}-MvaE-T_{XPR2} |
| Sq05 | Sq02, URA3:: T_{Lip1}-MvaS-P_{EXP1} + P_{TEFin}-MvaE-T_{XPR2} |
| Sq06 | Sq02, URA3:: T_{Lip1}-MvaS-P_{EXP1} + P_{TEFin}-MvaE-T_{XPR2} + T_{Lip1}-ERG20-P_{EXP1} + P_{TEFin}-ERG9-T_{XPR2} |
| Sq07 | Po1f-T-L, LYS5:: T_{Lip1}-ERG20-skl-P_{EXP1} + P_{TEFin}-ERG9-skl-T_{XPR2} |
| Sq08 | Sq03, URA3:: T_{Lip1}-ERG12-P_{EXP1} + P_{TEFin}-ERG8-T_{XPR2} + T_{Lip1}-ERG19-P_{EXP1} + P_{TEFin}-IDI-T_{XPR2} |
| Sq09 | Sq07, URA3:: T_{Lip1}-ERG12-skl-P_{EXP1} + P_{TEFin}-ERG8-skl-T_{XPR2} + T_{Lip1}-ERG19-skl-P_{EXP1} + P_{TEFin}- |
| | IDI-skl-T_{XPR2} |
| Sq10 | Po1f-T-L, URA3:: T_{Lip1}-ERG12-skl-P_{EXP1} + P_{TEFin}-ERG8-skl-T_{XPR2} + T_{Lip1}-ERG19-skl- |
| | P_{EXP1} + P_{TEFin}-IDI-skl-T_{XPR2}; LYS5:: T_{Lip1}-MvaS-skl-P_{EXP1} + P_{TEFin}-MvaE-skl-T_{XPR2} + T_{Lip1}- |
| | ERG20-skl-P_{EXP1} + P_{TEFin}-ERG9-skl-T_{XPR2} |
| Sq11 | Sq10, LEU2:: P_{TEFin}-ylTGL1-T_{XPR2} |
| Sq12 | Sq10, LEU2:: P_{TEFin}-ylTGL3-T_{XPR2} |
| Sq13 | Sq10, LEU2:: P_{TEFin}-ylTGL4-T_{XPR2} |
| Sq14 | Sq10, LEU2:: P_{TEFin}-tlTGL-T_{XPR2} |
| Sq15 | Sq14, TRP1:: P_{TEFin}-ACC1-T_{XPR2} + T_{Lip1}-DGA1-P_{EXP1} + P_{TEFin}-GPD1-T_{XPR2} |
| Sq16 | Sq06, TRP1:: P_{TEFin}-ACC1-T_{XPR2} + T_{Lip1}-DGA1-P_{EXP1} + P_{TEFin}-GPD1-T_{XPR2} |
| Sq17 | Sq15, hyg:: T_{Lip1}-6PGD-P_{EXP1} + P_{TEFin}-G6PD-T_{XPR2} |
| Sq18 | Sq15, hyg:: P_{TEFin}-PYC1-T_{XPR2} |
| Sq19 | Sq10, LEU2:: T_{Lip1}-tlTGL-P_{EXP1} + P_{TEFin}-PYC1-T_{XPR2} + T_{Lip1}-YHM2-P_{EXP1} + P_{TEFin}-MmACL- |
| | T_{XPR2}; TRP1:: P_{TEFin}-ACC1-T_{XPR2} + T_{Lip1}-DGA1-P_{EXP1} + P_{TEFin}-GPD1-T_{XPR2} |
| Sq20 | Sq19, hyg:: P_{TEFin}-POX1-T_{XPR2} |
| Sq21 | Sq19, hyg:: P_{TEFin}-POX2-T_{XPR2} |
| Sq22 | Sq19, hyg:: P_{TEFin}-POX3-T_{XPR2} |
| Sq23 | Sq19, hyg:: P_{TEFin}-POX4-T_{XPR2} |
| Sq24 | Sq19, hyg:: P_{TEFin}-POX5-T_{XPR2} |
| Sq25 | Sq19, hyg:: P_{TEFin}-POX6-T_{XPR2} |
| Sq26 | Sq19, hyg:: P_{TEFin}-POX2-T_{XPR2} + T_{Lip1}-MFE1-P_{EXP1} + P_{TEFin}-POT1-T_{XPR2} |
| Sq27 | Sq19, hyg:: T_{Lip1}-PEX10-P_{EXP1} + P_{TEFin}-POX2-T_{XPR2} + T_{Lip1}-MFE1-P_{EXP1} + P_{TEFin}-POT1-T_{XPR2} |
| Sq28 | Sq10, LEU2:: P_{TEFin}-SeACS-T_{XPR2} |
| Sq29 | Sq28, hyg:: T_{Lip1}-PEX10-P_{EXP1} + P_{TEFin}-POX2-T_{XPR2} + T_{Lip1}-MFE1-P_{EXP1} + P_{TEFin}-POT1-T_{XPR2} |

TABLE 2
Primers sequences.

| | | |
|---|---|---|
| Crispr-lys5-F | GGGTCGGCGCAGGTTGACGTTCTGACAGGCAAGAA | SEQ ID |
| | GTGGAGTTTTAGAGCTAGAAATAGC | NO: |
| Crispr-lys5-R | GCTATTTCTAGCTCTAAAACTCCACTTCTTGCCTGTC | 1 |
| | AGAACGTCAACCTGCGCCGACCC | |
| ERG10-1-F | ATGAACCAGATGCATAGCACCTACTCGACAGAAGAG | 2 |
| | ACCT | |
| ERG10-1-R | ACACAAGACATATCTACAGCATGCGACTCACTCTGC | 3 |
| | CCCG | |
| ERG10-2-F | TTTTGCAGTACTAACCGCAGGAGCCCGTCTACATTG | 4 |
| | T | |
| ERG10-2-R | GCAAGACCGGCAACGTGGGGCTAACACTTCTCAAC | 5 |
| | AATGA | |
| ERG13-F | ATGAACCAGATGCATAGCACCTACTGCTTGATCTCGT | 6 |
| | ACT | |
| ERG13-R | ACACAAGACATATCTACAGCATGTCGCAACCCCAGA | 7 |
| | ACGT | |
| tHMGR-F | TTTTGCAGTACTAACCGCAGACCCAGTCTGTGAAGG | 8 |
| | T | |
| tHMGR-R | GCAAGACCGGCAACGTGGGGCTATGACCGTATGCA | 9 |
| | AATAT | |
| MvaS-F | ATGAACCAGATGCATAGCACTTAGTTTCTGTAAGATC | 10 |
| | TGA | |
| MvaS-F (skl) | ATGAACCAGATGCATAGCACTTACAGCTTGGAGTTT | 11 |
| | CTGTAAGATCTGA | |
| MvaS-R | ACACAAGACATATCTACAGCATGACCATCGGTATCG | 12 |
| | ATAA | |
| MvaE-F | TTTTGCAGTACTAACCGCAGAAGACCGTTGTTATTAT | 13 |
| | CGA | |
| MvaE-R | GCAAGACCGGCAACGTGGGGTCATTGCTTCCTCAA | 14 |
| | ATCGT | |
| MvaE-R (skl) | GCAAGACCGGCAACGTGGGGTCACAGCTTGGATTG | 15 |
| | CTTCCTCAAATCGT | |
| ERG8-F | ATGAACCAGATGCATAGCACCTACTTGAACCCCTTC | 16 |
| | TCGA | |
| ERG8-F (skl) | ATGAACCAGATGCATAGCACCTACAGCTTGGACTTG | 17 |
| | AACCCCTTCTCGA | |
| ERG8-R | ACACAAGACATATCTACAGCATGACCACCTATTCGG | 18 |
| | CTCC | |
| ERG12-F | TTTTGCAGTACTAACCGCAGGACTACATCATTTCGGC | 19 |
| ERG12-R | GCAAGACCGGCAACGTGGGGCTAATGGGTCCAGGG | 20 |
| | ACCGA | |
| ERG12-R (skl) | GCAAGACCGGCAACGTGGGGCTACAGCTTGGAATG | 21 |
| | GGTCCAGGGACCGA | |
| IDI-F | ATGAACCAGATGCATAGCACCTACTTGATCCACCGC | 22 |
| | CGAA | |
| IDI-F (skl) | ATGAACCAGATGCATAGCACCTACAGCTTGGACTTG | 23 |
| | ATCCACCGCCGAA | |
| IDI-R | ACACAAGACATATCTACAGCATGACGACGTCTTACA | 24 |
| | GCGA | |
| ERG19-F | TTTTGCAGTACTAACCGCAGATCCACCAGGCCTCCA | 25 |
| | C | |
| ERG19-R | GCAAGACCGGCAACGTGGGGCTACTTGCTGTTCTTC | 26 |
| | AGAG | |
| ERG19-R (skl) | GCAAGACCGGCAACGTGGGGCTACAGCTTGGACTT | 27 |
| | GCTGTTCTTCAGAG | |
| ERG20-F | ATGAACCAGATGCATAGCACCTACTTCTGTCGCTTGT | 28 |
| | AAA | |
| ERG20-F (skl) | ATGAACCAGATGCATAGCACCTACAGCTTGGACTTC | 29 |
| | TGTCGCTTGTAAA | |
| ERG20-R | ACACAAGACATATCTACAGCATGTCCAAGGCGAAAT | 30 |
| | TCGA | |
| ERG9-F | TTTTGCAGTACTAACCGCAGGGAAAACTCATCGAAC | 31 |
| | TGCT | |
| ERG9-R | GCAAGACCGGCAACGTGGGGCTAATCTCTCAGAGG | 32 |
| | AAACA | |
| ERG9-R (skl) | GCAAGACCGGCAACGTGGGGCTACAGCTTGGAATC | 33 |
| | TCTCAGAGGAAACA | |
| yITGL1-F | TTTTGCAGTACTAACCGCAGCACGTCCCCGTTCTAG | 34 |
| | GACGTC | |
| yITGL1-R | GCAAGACCGGCAACGTGGGGTCAAGGGTTGAGAGT | 35 |
| | GCCACTC | |
| ylTGL3-F | TTTTGCAGTACTAACCGCAGAAAAGCCGCGTGGCCG | 36 |
| | TTGTCT | |
| ylTGL3-R | GCAAGACCGGCAACGTGGGGCTAGTTTTGTCGCTTG | 37 |
| | GTCTGG | |
| ylTGL4-F | TTTTGCAGTACTAACCGCAGTTCACCTCCAGAGTTT | 38 |
| | CCGAAG | |
| ylTGL4-R | GCAAGACCGGCAACGTGGGGTTAGCACGAGTCAGA | 39 |
| | ACAGTTC | |
| tlTGL-F | TTTTGCAGTACTAACCGCAGCGATCCTCCCTGGTCCT | 40 |
| | GTT | |
| tlTGL-R | GCAAGACCGGCAACGTGGGGTTACAGGCAGGTGCC | 41 |
| | GATCA | |
| ACC1-F | TTTTGCAGTACTAACCGCAGCGACTGCAATTGAGGA | 42 |
| | CACTAACAC | |
| ACC1-R | GCAAGACCGGCAACGTGGGGTCACAACCCCTTGAG | 43 |
| | CAGCTCA | |
| DGA1-F | ATGAACCAGATGCATAGCACTTACTCAATCATTCGGA | 44 |
| | ACT | |
| DGA1-R | ACACAAGACATATCTACAGCATGACTATCGACTCAC | 45 |
| | AATA | |
| GPD1-F | TTTTGCAGTACTAACCGCAGAGCGCTCTACTTCGAT | 46 |
| | CGT | |
| GPD1-R | GCAAGACCGGCAACGTGGGGCTAGTTGGCGTGGTA | 47 |
| | AAGAATC | |
| PYC1-F | TTTTGCAGTACTAACCGCAGTCCAACGTTCCTGAGA | 48 |
| | CCAA | |
| PYC1-R | GCAAGACCGGCAACGTGGGGTTAAGCCCGCACAAT | 49 |
| | CTTGC | |
| YHM2-F | ATGAACCAGATGCATAGCACCTAGTGCTTACCAACA | 50 |
| | GGTC | |
| YHM2-R | ACACAAGACATATCTACAGCATGGGTGCTGCTAACC | 51 |
| | TCAA | |
| MmACL-F | TTTTGCAGTACTAACCGCAGTCTGCCAAGGCCATCT | 52 |
| | CTGA | |
| MmACL-R | GCAAGACCGGCAACGTGGGGTTACATAGACATGTGC | 53 |
| | TCGG | |
| POX1-F | TTTTGCAGTACTAACCGCAGGCCAAGGAGCGAGGT | 54 |
| | AAGA | |
| POX1-R | GCAAGACCGGCAACGTGGGGTCACTCATCGAGATC | 55 |
| | GCAAATT | |
| POX2-F | TTTTGCAGTACTAACCGCAGAACCCCAACAACACTG | 56 |
| | GCA | |
| POX2-R | GCAAGACCGGCAACGTGGGGCTATTCCTCATCAAGC | 57 |
| | TCGCAA | |
| POX3-F | TTTTGCAGTACTAACCGCAGATCTCCCCCAACCTCA | 58 |
| | CAG | |
| POX3-R | GCAAGACCGGCAACGTGGGGCTATTCCTCGTCCAGC | 59 |
| | TCGCAA | |
| POX4-F | TTTTGCAGTACTAACCGCAGATCACCCCAAACCCCG | 60 |
| | CTA | |
| POX4-R | GCAAGACCGGCAACGTGGGGTTACTGAATATCCTCG | 61 |
| | GGCTCC | |
| POX5-F | TTTTGCAGTACTAACCGCAGAACAACAACCCCACCA | 62 |
| | ACG | |
| POX5-R | GCAAGACCGGCAACGTGGGGCTACTCGTCCAGGTC | 63 |
| | GCAAATC | |
| POX6-F | TTTTGCAGTACTAACCGCAGCTCTCTCAACAGTCCC | 64 |
| | TCA | |
| POX6-R | GCAAGACCGGCAACGTGGGGCTACTCATCCTCAAG | 65 |
| | AGAGCAA | |
| MFE1-F | ATGAACCAGATGCATAGCACTTAGAGCTTAGCATCC | 66 |
| | TTGG | |
| MFE1-R | ACACAAGACATATCTACAGCATGTCTGGAGAACTAA | 67 |
| | GATA | |
| POT1-F | TTTTGCAGTACTAACCGCAGGACCGACTTAACAACC | 68 |
| | TCG | |
| POT1-R | GCAAGACCGGCAACGTGGGGTTACTCGGCAACAAC | 69 |
| | CAGAG | |
| PEX10-F | ATGAACCAGATGCATAGCACTTATCTGATAGGCAAC | 70 |
| | AAGT | |
| PEX10-R | ACACAAGACATATCTACAGCATGTGGGGAAGTTCAC | 71 |
| | ATGC | |
| SeACS-F | TTTTGCAGTACTAACCGCAGTCTCAGACCCACAAGC | 72 |
| | ACGC | |
| SeACS-R | GCAAGACCGGCAACGTGGGGTTAAGAGGGCATAGC | 73 |
| | AATGG | |
| 6PGD-F | ATGAACCAGATGCATAGCACTTAAGCATCGTAAGTG | 74 |
| | GAAG | |
| 6PGD-R | ACACAAGACATATCTACAGCATGACTGACACTTCAA | 75 |
| | ACAT | |
| G6PD-F | TTTTGCAGTACTAACCGCAGACTGGCACCTTACCCA | 76 |
| | AGT | |
| G6PD-R | GCAAGACCGGCAACGTGGGGTCACGAGGAGCCCTT | 77 |
| | GGTGACA | |

### REFERENCES

1. Y. Chen, J. Nielsen, Advances in metabolic pathway and strain engineering paving the way for sustainable production of chemical building blocks. Curr. Opin. Biotechnol. 24, 965-972 (2013).
2. P. P. Peralta-Yahya, F. Zhang, S. B. Del Cardayre, J. D. Keasling, Microbial engineering for the production of advanced biofuels. Nature 488, 320-328 (2012).
3. Y. Liu, J. Nielsen, Recent trends in metabolic engineering of microbial chemical factories. Curr. Opin. Biotechnol. 60, 188-197 (2019).
4. C. E. Vickers, T. C. Williams, B. Peng, J. Cherry, Recent advances in synthetic biology for engineering isoprenoid production in yeast. Curr. Opin. Chem. Biol. 40, 47-56 (2017).
5. J. Nielsen, J. D. Keasling, Engineering cellular metabolism. Cell 164, 1185-1197 (2016).
6. L. Dong et al., Co-expression of squalene epoxidases with triterpene cyclases boosts production of triterpenoids in plants and yeast. Metab. Eng. 49, 1-12 (2018).
7. D.-K. Ro et al., Production of the antimalarial drug precursor artemisinic acid in engineered yeast. Nature 440, 940-943 (2006).
8. C. J. Paddon et al., High-level semi-synthetic production of the potent antimalarial artemisinin. Nature 496, 528-532 (2013).
9. G.-S. Liu et al., The yeast peroxisome: A dynamic storage depot and subcellular factory for squalene overproduction. Metab. Eng. 57, 151-161 (2020).
10. C. Ignea et al., Orthogonal monoterpenoid biosynthesis in yeast constructed on an isomeric substrate. Nat. Commun. 10, 3799 (2019).
11. A. Chou, S. H. Lee, F. Zhu, J. M. Clomburg, R. Gonzalez, An orthogonal metabolic framework for one-carbon utilization. Nature Metabolism 3, 1385-1399 (2021).
12. R. Thimmappa, K. Geisler, T. Louveau, P. O'Maille, A. Osbourn, Triterpene biosynthesis in plants. Annu. Rev. Plant Biol. 65, 225-257 (2014).
13. J. D. Connolly, R. A. Hill, Triterpenoids. Nat. Prod. Rep. 27, 79-132 (2010).
14. K. Paramasivan, S. Mutturi, Recent advances in the microbial production of squalene. World J. Microbiol. Biotechnol. 38, 1-21 (2022).
15. M. Tateno et al., Synthetic Biology-derived triterpenes as efficacious immunomodulating adjuvants. Sci. Rep. 10, 1-11 (2020).
16. R. Thimmappa, K. Geisler, T. Louveau, P. O'Maille, A. Osbourn, Triterpene biosynthesis in plants. Annu. Rev. Plant Biol. 65 (2014).
17. N. Gohil, G. Bhattacharjee, K. Khambhati, D. Braddick, V. Singh, Engineering strategies in microorganisms for the enhanced production of squalene: advances, challenges and opportunities. Frontiers in bioengineering and biotechnology 7, 50 (2019).
18. Z.-T. Zhu et al., Metabolic compartmentalization in yeast mitochondria: Burden and solution for squalene overproduction. Metab. Eng. 68, 232-245 (2021).
19. L. J. Wei et al., Improved squalene production through increasing lipid contents in Saccharomyces cerevisiae. Biotechnol. Bioeng. 115, 1793-1800 (2018).
20. A. Rasool, M. S. Ahmed, C. Li, Overproduction of squalene synergistically downregulates ethanol production in Saccharomyces cerevisiae. Chem. Eng. Sci. 152, 370-380 (2016).
21. J. Y. Han, S. H. Seo, J. M. Song, H. Lee, E.-S. Choi, High-level recombinant production of squalene using selected Saccharomyces cerevisiae strains. J. Ind. Microbiol. Biotechnol. 45, 239-251 (2018).
22. W. Xu et al., Improving squalene production by enhancing the NADPH/NADP+ ratio, modifying the isoprenoid-feeding module and blocking the menaquinone pathway in Escherichia coli. Biotechnol. Biofuels 12, 1-9 (2019).
23. J.-J. Pan et al., Biosynthesis of squalene from farnesyl diphosphate in bacteria: three steps catalyzed by three enzymes. ACS central science 1, 77-82 (2015).
24. A. Katabami et al., Production of squalene by squalene synthases and their truncated mutants in Escherichia coli. J. Biosci. Bioeng. 119, 165-171 (2015).
25. S. Gao et al., Iterative integration of multiple-copy pathway genes in Yarrowia lipolytica for heterologous β-carotene production. Metab. Eng. 41, 192-201 (2017).
26. Y.-Y. Huang et al., Enhanced squalene biosynthesis in Yarrowia lipolytica based on metabolically engineered acetyl-CoA metabolism. J. Biotechnol. 281, 106-114 (2018).
27. X. Luo et al., Complete biosynthesis of cannabinoids and their unnatural analogues in yeast. Nature 567, 123-126 (2019).
28. S. Dussdaux, W. T. Wajn, Y. Liu, C. Ignea, S. C. Kampranis, Transforming yeast peroxisomes into microfactories for the efficient production of high-value isoprenoids. Proc. Natl. Acad. Sci. USA 117, 31789-31799 (2020).
29. Y. Ma et al., Removal of lycopene substrate inhibition enables high carotenoid productivity in Yarrowia lipolytica. Nat. Commun. 13, 572 (2022).
30. H. Liu, M. Marsafari, F. Wang, L. Deng, P. Xu, Engineering acetyl-CoA metabolic shortcut for eco-friendly production of polyketides triacetic acid lactone in Yarrowia lipolytica. Metab. Eng. 56, 60-68 (2019).
31. M. T. Alam et al., The self-inhibitory nature of metabolic networks and its alleviation through compartmentalization. Nat. Commun. 8, 1-13 (2017).
32. A. A. Sibirny, Yeast peroxisomes: structure, functions and biotechnological opportunities. FEMS Yeast Res. 16 (2016).
33. K. Yang et al., Subcellular engineering of lipase dependent pathways directed towards lipid related organelles for highly effectively compartmentalized biosynthesis of triacylglycerol derived products in Yarrowia lipolytica. Metab. Eng. 55, 231-238 (2019).
34. M. Tai, G. Stephanopoulos, Engineering the push and pull of lipid biosynthesis in oleaginous yeast Yarrowia lipolytica for biofuel production. Metab. Eng. 15, 1-9 (2013).
35. T. Dulermo, J.-M. Nicaud, Involvement of the G3P shuttle and β-oxidation pathway in the control of TAG synthesis and lipid accumulation in Yarrowia lipolytica. Metab. Eng. 13, 482-491 (2011).
36. M. Larroude et al., A synthetic biology approach to transform Yarrowia lipolytica into a competitive biotechnological producer of β-carotene. Biotechnol. Bioeng. 115, 464-472 (2018).
37. K. Qiao, T. M. Wasylenko, K. Zhou, P. Xu, G. Stephanopoulos, Lipid production in Yarrowia lipolytica is maximized by engineering cytosolic redox metabolism. Nat. Biotechnol. 35, 173 (2017).
38. H. Zhang et al., Enhanced lipid accumulation in the yeast Yarrowia lipolytica by over-expression of ATP: citrate lyase from Mus musculus. J. Biotechnol. 192, 78-84 (2014).
39. E. Y. Yuzbasheva et al., The mitochondrial citrate carrier in Yarrowia lipolytica: its identification, characterization and functional significance for the production of citric acid. Metab. Eng. 54, 264-274 (2019).
40. A. Beopoulos et al., Control of lipid accumulation in the yeast Yarrowia lipolytica. Appl. Environ. Microbiol. 74, 7779-7789 (2008).
41. P. Kasaragod et al., Structural enzymology comparisons of multifunctional enzyme, type-1 (MFE 1): the flexibility of its dehydrogenase part. FEBS Open bio 7, 1830-1842 (2017).
42. J. J. Smith, T. W. Brown, G. A. Eitzen, R. A. Rachubinski, Regulation of peroxisome size and number by fatty acid β-oxidation in the yeast Yarrowia lipolytica. J. Biol. Chem. 275, 20168-20178 (2000).
43. V. J. Starai, J. G. Gardner, J. C. Escalante-Semerena, Residue Leu-641 of acetyl-CoA synthetase is critical for the acetylation of residue Lys-609 by the protein acetyltransferase enzyme of Salmonella enterica. J. Biol. Chem. 280, 26200-26205 (2005).
44. J. O. Park et al., Synergistic substrate cofeeding stimulates reductive metabolism. Nature Metabolism 1, 643-651 (2019).
45. J. M. Gancedo, Yeast carbon catabolite repression. Microbiol. Mol. Biol. Rev. 62, 334-361 (1998).
46. T. Yu et al., Reprogramming Yeast Metabolism from Alcoholic Fermentation to Lipogenesis. Cell (2018).
47. J. Xu, N. Liu, K. Qiao, S. Vogg, G. Stephanopoulos, Application of metabolic controls for the maximization of lipid production in semicontinuous fermentation. Proc. Natl. Acad. Sci. USA 114, E5308-E5316 (2017).

What is claimed is:

1. A modified yeast cell, comprising:
a first set of heterologous polynucleotides encoding a first set of enzymes of the mevalonate pathway, wherein one or more of the first set of enzymes of the mevalonate pathway is/are linked to a peroxisomal targeting signal;
a first heterologous polynucleotide encoding a lipase; and
a second set of heterologous polynucleotides encoding a second set of enzymes having triacylglycerol synthesis activity.

2. The modified yeast cell of claim 1, further comprising a third set of heterologous polynucleotides encoding a third set of enzymes having acetyl-CoA synthesis activity.

3. The modified yeast cell of claim 1, further comprising a fourth set of heterologous polynucleotides encoding a fourth set of enzymes having β-oxidation activity.

4. The modified yeast cell of claim 1, wherein the first set of enzymes of the mevalonate pathway comprises ERG10, ERG13, tHMGR, mvaE, mvaS, ERG12, ERG8, ERG19, IDI, ERG20, ERG9, or any combination thereof.

5. The modified yeast cell of claim 1, wherein each enzyme of the first set of enzymes of the mevalonate pathway comprises a peroxisomal targeting signal.

6. The modified yeast cell of claim 4, wherein ERG20 and ERG9 each comprise a peroxisomal targeting signal.

7. The modified yeast cell of claim 1, wherein the peroxisomal targeting signal is at the C-terminus of the one or more of the first set of enzymes of the mevalonate pathway.

8. The modified yeast cell of claim 1, wherein the peroxisomal targeting signal is at the N-terminus of the one or more of the first set of enzymes of the mevalonate pathway.

9. The modified yeast cell of claim 1, wherein the peroxisomal targeting signal is peroxisomal targeting sequence 1 (PTS1), peroxisomal targeting sequence 2 (PTS2), or peroxisomal targeting sequence 3 (PTS3).

10. The modified yeast cell of claim 9, wherein the PTS1 is the amino acid sequence serine-lysine-leucine (SKL).

11. The modified yeast cell of claim 1, wherein the lipase is a triacylglycerol lipase derived from Thermomyces lanuginosus.

12. The modified yeast cell of claim 1, wherein the second set of enzymes having triacylglycerol synthesis activity comprises acetyl-CoA carboxylase (ACC1), diacylglycerol acyltransferase (DGA1), NAD+-dependent G3P dehydrogenase (GPD1), or any combination thereof.

13. The modified yeast cell of claim 2, wherein the third set of enzymes having acetyl-CoA synthesis activity comprises pyruvate carboxylase (PYC1), ATP:citrate lyase (ACL), citrate carrier YHM2, acetyl-CoA synthetase, or any combination thereof.

14. The modified yeast cell of claim 13, wherein the ACL is derived from Mus musculus.

15. The modified yeast cell of claim 13, wherein the acetyl-CoA synthetase is derived from Salmonella enterica.

16. The modified yeast cell of claim 15, wherein the acetyl-CoA synthetase comprises an L641P amino acid substitution mutation relative to a wild-type acetyl-CoA synthetase.

17. The modified yeast cell of claim 3, wherein the fourth set of enzymes having β-oxidation activity comprises POX1, POX2, POX3, POX4, POX5, POX6, multifunctional β-oxidation protein (MFE1), 3-ketoacyl-CoA thiolase (POT1), or any combination thereof.

18. The modified yeast cell of claim 1, wherein the modified yeast cell is an oleaginous yeast cell.

19. The modified yeast cell of claim 18, wherein the oleaginous yeast cell is a Yarrowia cell.

20. The modified yeast cell of claim 19, wherein the Yarrowia cell is Yarrowia lipolytica.

21. A method of producing squalene, comprising: culturing a modified yeast cell according to claim 1 in a culture medium for a sufficient time to produce squalene in the peroxisome of the modified yeast cell.

22. The method of claim 21, further comprising extracting squalene from the modified yeast cell culture.

23. The method of claim 21, wherein the culture medium comprises yeast extract, peptone, glucose, and an aqueous buffer.

24. The method of claim 21, wherein the culture medium comprises acetate.

25. The method of claim 21, wherein the culture medium comprises acetate and a limited amount of one or more sugars.

26. The method of claim 25, wherein the one or more sugars comprise glucose, fructose, and/or xylose.

27. The method of claim 21, wherein the culture medium comprises a buffer, optionally wherein the buffer is phosphate-buffered saline.

28. The method of claim 21, wherein the sufficient time is at least 6 hours, at least 12 hours, at least 24 hours, at least 48 hours, at least 72 hours, at least 96 hours, or at least 192 hours.

29. The method of claim 21, wherein at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is in the culture medium.

30. The method of claim 21, wherein at least 0.1 g/L, at least 0.2 g/L, at least 0.3 g/L, at least 0.4 g/L, at least 0.5 g/L, at least 1.0 g/L, at least 1.5 g/L, or more than 1.5 g/L of squalene is extracted from the modified yeast cell culture.

31. The method of claim 21, wherein at least at least 25 mg/g dry cell weight (DCW), at least 50 mg/g DCW, at least 100 mg/g DCW, at least 150 mg/g DCW, or more than 150 mg/g DCW of squalene is extracted from the modified yeast cell culture.


SEQUENCE LISTING (78 sequences)

SEQ ID NO: 1 - DNA, 60 bp, Synthetic construct
  source 1..60: mol_type = other DNA
gctatttctagctctaaaactccacttcttgcctgtcagaacgtcaacctgcgccgaccc

SEQ ID NO: 2 - DNA, 40 bp, Synthetic construct
  source 1..40: mol_type = other DNA
atgaaccagatgcatagcacctactcgacagaagagacct

SEQ ID NO: 3 - DNA, 40 bp, Synthetic construct
  source 1..40: mol_type = other DNA
acacaagacatatctacagcatgcgactcactctgccccg

SEQ ID NO: 4 - DNA, 37 bp, Synthetic construct
  source 1..37: mol_type = other DNA
ttttgcagtactaaccgcaggagcccgtctacattgt

SEQ ID NO: 5 - DNA, 40 bp, Synthetic construct
  source 1..40: mol_type = other DNA
gcaagaccggcaacgtggggctaacacttctcaacaatga

SEQ ID NO: 6 - DNA, 40 bp, Synthetic construct
  source 1..40: mol_type = other DNA
atgaaccagatgcatagcacctactgcttgatctcgtact

SEQ ID NO: 7 - DNA, 40 bp, Synthetic construct
  source 1..40: mol_type = other DNA
acacaagacatatctacagcatgtcgcaaccccagaacgt

SEQ ID NO: 8 - DNA, 37 bp, Synthetic construct
  source 1..37: mol_type = other DNA
ttttgcagtactaaccgcagacccagtctgtgaaggt

SEQ ID NO: 9 - DNA, 40 bp, Synthetic construct
  source 1..40: mol_type = other DNA
gcaagaccggcaacgtggggctatgaccgtatgcaaatat

SEQ ID NO: 10 - DNA, 40 bp, Synthetic construct
  source 1..40: mol_type = other DNA
atgaaccagatgcatagcacttagtttctgtaagatctga

SEQ ID NO: 11 - DNA, 49 bp, Synthetic construct
  source 1..49: mol_type = other DNA
atgaaccagatgcatagcacttacagcttggagtttctgtaagatctga

SEQ ID NO: 12 - DNA, 40 bp, Synthetic construct
  source 1..40: mol_type = other DNA
acacaagacatatctacagcatgaccatcggtatcgataa

SEQ ID NO: 13 - DNA, 40 bp, Synthetic construct
  source 1..40: mol_type = other DNA
ttttgcagtactaaccgcagaagaccgttgttattatcga

SEQ ID NO: 14 - DNA, 40 bp, Synthetic construct
  source 1..40: mol_type = other DNA
gcaagaccggcaacgtggggtcattgcttcctcaaatcgt

SEQ ID NO: 15 - DNA, 49 bp, Synthetic construct
  source 1..49: mol_type = other DNA
gcaagaccggcaacgtggggtcacagcttggattgcttcctcaaatcgt

SEQ ID NO: 16 - DNA, 40 bp, Synthetic construct
  source 1..40: mol_type = other DNA
atgaaccagatgcatagcacctacttgaaccccttctcga

SEQ ID NO: 17 - DNA, 49 bp, Synthetic construct
  source 1..49: mol_type = other DNA
atgaaccagatgcatagcacctacagcttggacttgaaccccttctcga

SEQ ID NO: 18 - DNA, 40 bp, Synthetic construct
  source 1..40: mol_type = other DNA
acacaagacatatctacagcatgaccacctattcggctcc

SEQ ID NO: 19 - DNA, 37 bp, Synthetic construct
  source 1..37: mol_type = other DNA
ttttgcagtactaaccgcaggactacatcatttcggc

SEQ ID NO: 20 - DNA, 40 bp, Synthetic construct
  source 1..40: mol_type = other DNA
gcaagaccggcaacgtggggctaatgggtccagggaccga

SEQ ID NO: 21 - DNA, 49 bp, Synthetic construct
  source 1..49: mol_type = other DNA
gcaagaccggcaacgtggggctacagcttggaatgggtccagggaccga

SEQ ID NO: 22 - DNA, 40 bp, Synthetic construct
  source 1..40: mol_type = other DNA
atgaaccagatgcatagcacctacttgatccaccgccgaa

SEQ ID NO: 23 - DNA, 49 bp, Synthetic construct
  source 1..49: mol_type = other DNA
atgaaccagatgcatagcacctacagcttggacttgatccaccgccgaa

SEQ ID NO: 24 - DNA, 40 bp, Synthetic construct
  source 1..40: mol_type = other DNA
acacaagacatatctacagcatgacgacgtcttacagcga

SEQ ID NO: 25 - DNA, 37 bp, Synthetic construct
  source 1..37: mol_type = other DNA
ttttgcagtactaaccgcagatccaccaggcctccac

SEQ ID NO: 26 - DNA, 40 bp, Synthetic construct
  source 1..40: mol_type = other DNA
gcaagaccggcaacgtggggctacttgctgttcttcagag

SEQ ID NO: 27 - DNA, 49 bp, Synthetic construct
  source 1..49: mol_type = other DNA
gcaagaccggcaacgtggggctacagcttggacttgctgttcttcagag

SEQ ID NO: 28 - DNA, 40 bp, Synthetic construct
  source 1..40: mol_type = other DNA
atgaaccagatgcatagcacctacttctgtcgcttgtaaa

SEQ ID NO: 29 - DNA, 49 bp, Synthetic construct
  source 1..49: mol_type = other DNA
atgaaccagatgcatagcacctacagcttggacttctgtcgcttgtaaa

SEQ ID NO: 30 - DNA, 40 bp, Synthetic construct
  source 1..40: mol_type = other DNA
acacaagacatatctacagcatgtccaaggcgaaattcga

SEQ ID NO: 31 - DNA, 40 bp, Synthetic construct
  source 1..40: mol_type = other DNA
ttttgcagtactaaccgcagggaaaactcatcgaactgct

SEQ ID NO: 32 - DNA, 40 bp, Synthetic construct
  source 1..40: mol_type = other DNA
gcaagaccggcaacgtggggctaatctctcagaggaaaca

SEQ ID NO: 33 - DNA, 49 bp, Synthetic construct
  source 1..49: mol_type = other DNA
gcaagaccggcaacgtggggctacagcttggaatctctcagaggaaaca

SEQ ID NO: 34 - DNA, 42 bp, Synthetic construct
  source 1..42: mol_type = other DNA
ttttgcagtactaaccgcagcacgtccccgttctaggacgtc

SEQ ID NO: 35 - DNA, 42 bp, Synthetic construct
  source 1..42: mol_type = other DNA
gcaagaccggcaacgtggggtcaagggttgagagtgccactc

SEQ ID NO: 36 - DNA, 42 bp, Synthetic construct
  source 1..42: mol_type = other DNA
ttttgcagtactaaccgcagaaaagccgcgtggccgttgtct

SEQ ID NO: 37 - DNA, 42 bp, Synthetic construct
  source 1..42: mol_type = other DNA
gcaagaccggcaacgtggggctagttttgtcgcttggtctgg

SEQ ID NO: 38 - DNA, 42 bp, Synthetic construct
  source 1..42: mol_type = other DNA
ttttgcagtactaaccgcagttcacctccagagtttccgaag

SEQ ID NO: 39 - DNA, 42 bp, Synthetic construct
  source 1..42: mol_type = other DNA
gcaagaccggcaacgtggggttagcacgagtcagaacagttc

SEQ ID NO: 40 - DNA, 40 bp, Synthetic construct
  source 1..40: mol_type = other DNA
ttttgcagtactaaccgcagcgatcctccctggtcctgtt

SEQ ID NO: 41 - DNA, 40 bp, Synthetic construct
  source 1..40: mol_type = other DNA
gcaagaccggcaacgtggggttacaggcaggtgccgatca

SEQ ID NO: 42 - DNA, 45 bp, Synthetic construct
  source 1..45: mol_type = other DNA
ttttgcagtactaaccgcagcgactgcaattgaggacactaacac

SEQ ID NO: 43 - DNA, 42 bp, Synthetic construct
  source 1..42: mol_type = other DNA
gcaagaccggcaacgtggggtcacaaccccttgagcagctca

SEQ ID NO: 44 - DNA, 40 bp, Synthetic construct
  source 1..40: mol_type = other DNA
atgaaccagatgcatagcacttactcaatcattcggaact

SEQ ID NO: 45 - DNA, 40 bp, Synthetic construct
  source 1..40: mol_type = other DNA
acacaagacatatctacagcatgactatcgactcacaata

SEQ ID NO: 46 - DNA, 39 bp, Synthetic construct
  source 1..39: mol_type = other DNA
ttttgcagtactaaccgcagagcgctctacttcgatcgt

SEQ ID NO: 47 - DNA, 42 bp, Synthetic construct
  source 1..42: mol_type = other DNA
gcaagaccggcaacgtggggctagttggcgtggtaaagaatc

SEQ ID NO: 48 - DNA, 40 bp, Synthetic construct
  source 1..40: mol_type = other DNA
ttttgcagtactaaccgcagtccaacgttcctgagaccaa

SEQ ID NO: 49 - DNA, 40 bp, Synthetic construct
  source 1..40: mol_type = other DNA
gcaagaccggcaacgtggggttaagcccgcacaatcttgc

SEQ ID NO: 50 - DNA, 40 bp, Synthetic construct
  source 1..40: mol_type = other DNA
atgaaccagatgcatagcacctagtgcttaccaacaggtc

SEQ ID NO: 51 - DNA, 40 bp, Synthetic construct
  source 1..40: mol_type = other DNA
acacaagacatatctacagcatgggtgctgctaacctcaa

SEQ ID NO: 52 - DNA, 40 bp, Synthetic construct
  source 1..40: mol_type = other DNA
ttttgcagtactaaccgcagtctgccaaggccatctctga

SEQ ID NO: 53 - DNA, 40 bp, Synthetic construct
  source 1..40: mol_type = other DNA
gcaagaccggcaacgtggggttacatagacatgtgctcgg

SEQ ID NO: 54 - DNA, 39 bp, Synthetic construct
  source 1..39: mol_type = other DNA
ttttgcagtactaaccgcaggccaaggagcgaggtaaga

SEQ ID NO: 55 - DNA, 42 bp, Synthetic construct
  source 1..42: mol_type = other DNA
gcaagaccggcaacgtggggtcactcatcgagatcgcaaatt

SEQ ID NO: 56 - DNA, 39 bp, Synthetic construct
  source 1..39: mol_type = other DNA
ttttgcagtactaaccgcagaaccccaacaacactggca

SEQ ID NO: 57 - DNA, 42 bp, Synthetic construct
  source 1..42: mol_type = other DNA
gcaagaccggcaacgtggggctattcctcatcaagctcgcaa

SEQ ID NO: 58 - DNA, 39 bp, Synthetic construct
  source 1..39: mol_type = other DNA
ttttgcagtactaaccgcagatctcccccaacctcacag

SEQ ID NO: 59 - DNA, 42 bp, Synthetic construct
  source 1..42: mol_type = other DNA
gcaagaccggcaacgtggggctattcctcgtccagctcgcaa

SEQ ID NO: 60 - DNA, 39 bp, Synthetic construct
  source 1..39: mol_type = other DNA
ttttgcagtactaaccgcagatcaccccaaaccccgcta

SEQ ID NO: 61 - DNA, 42 bp, Synthetic construct
  source 1..42: mol_type = other DNA
gcaagaccggcaacgtggggttactgaatatcctcgggctcc

SEQ ID NO: 62 - DNA, 39 bp, Synthetic construct
  source 1..39: mol_type = other DNA
ttttgcagtactaaccgcagaacaacaaccccaccaacg

SEQ ID NO: 63 - DNA, 42 bp, Synthetic construct
  source 1..42: mol_type = other DNA
gcaagaccggcaacgtggggctactcgtccaggtcgcaaatc

SEQ ID NO: 64 - DNA, 39 bp, Synthetic construct
  source 1..39: mol_type = other DNA
ttttgcagtactaaccgcagctctctcaacagtccctca

SEQ ID NO: 65 - DNA, 42 bp, Synthetic construct
  source 1..42: mol_type = other DNA
gcaagaccggcaacgtggggctactcatcctcaagagagcaa

SEQ ID NO: 66 - DNA, 40 bp, Synthetic construct
  source 1..40: mol_type = other DNA
atgaaccagatgcatagcacttagagcttagcatccttgg

SEQ ID NO: 67 - DNA, 40 bp, Synthetic construct
  source 1..40: mol_type = other DNA
acacaagacatatctacagcatgtctggagaactaagata

SEQ ID NO: 68 - DNA, 39 bp, Synthetic construct
  source 1..39: mol_type = other DNA
ttttgcagtactaaccgcaggaccgacttaacaacctcg

SEQ ID NO: 69 - DNA, 40 bp, Synthetic construct
  source 1..40: mol_type = other DNA
gcaagaccggcaacgtggggttactcggcaacaaccagag

SEQ ID NO: 70 - DNA, 40 bp, Synthetic construct
  source 1..40: mol_type = other DNA
atgaaccagatgcatagcacttatctgataggcaacaagt

SEQ ID NO: 71 - DNA, 40 bp, Synthetic construct
  source 1..40: mol_type = other DNA
acacaagacatatctacagcatgtggggaagttcacatgc

SEQ ID NO: 72 - DNA, 40 bp, Synthetic construct
  source 1..40: mol_type = other DNA
ttttgcagtactaaccgcagtctcagacccacaagcacgc

SEQ ID NO: 73 - DNA, 40 bp, Synthetic construct
  source 1..40: mol_type = other DNA
gcaagaccggcaacgtggggttaagagggcatagcaatgg

SEQ ID NO: 74 - DNA, 40 bp, Synthetic construct
  source 1..40: mol_type = other DNA
atgaaccagatgcatagcacttaagcatcgtaagtggaag

SEQ ID NO: 75 - DNA, 40 bp, Synthetic construct
  source 1..40: mol_type = other DNA
acacaagacatatctacagcatgactgacacttcaaacat

SEQ ID NO: 76 - DNA, 39 bp, Synthetic construct
  source 1..39: mol_type = other DNA
ttttgcagtactaaccgcagactggcaccttacccaagt

SEQ ID NO: 77 - DNA, 42 bp, Synthetic construct
  source 1..42: mol_type = other DNA
gcaagaccggcaacgtggggtcacgaggagcccttggtgaca

SEQ ID NO: 78 - DNA, 20 bp, Synthetic construct
  source 1..20: mol_type = other DNA
tctgacaggcaagaagtgga